(19) United States
(12) Patent Application Publication
(10) Pub. No.: US 2004/0052768 A1
(43) Pub. Date: Mar. 18, 2004

(54) Vascularised tissue graft

(76) Inventors: Wayne A. Morrison, Victoria (AU); Aurora Messina, Victoria (AU); Kenneth R. Knight, Victoria (AU); Anthony J. Penington, Victoria (AU)

Correspondence Address: KNOBBE MARTENS OLSON & BEAR LLP, 2040 MAIN STREET, FOURTEENTH FLOOR, IRVINE, CA, 92614, US

(21) Appl. No.: 10/362,243

(22) PCT Filed: Aug. 21, 2001
(86) PCT No.: PCT/AU01/01031

(30) Foreign Application Priority Data
Aug. 21, 2000 (AU) PQ 9553

(51) Int. Cl. A61K 45/00

(52) U.S. Cl. 424/93.7

(57) ABSTRACT

A method of producing vascularised tissue utilizing a vascular pedicle enclosed in a chamber and implanted in a donor is provided. A vascularised tissue graft suitable for transplantation is also provided. The invention also encompasses a method of repairing a tissue deficit using a vascularised tissue graft.

This invention relates to the fields of tissue engineering and transplantation, and particularly to the generation of vascularised tissue.

### BACKGROUND OF THE INVENTION

Tissue engineering utilising homologous starting material offers the prospect of replacing missing or non-functioning body parts with newly created, living tissue. It has the potential to minimise loss of tissue and resultant pain from the donor site experienced in conventional reconstructive surgery or to recreate specialized tissue for which there is no donor site, while obviating the long-term immunosuppression required for heterologous transplantation.

It combines the techniques of tissue culture, the creation of bio-compatible materials and the manipulation of angiogenesis in order to create new, vascularised tissue to replace damaged tissue or tissue which is congenitally absent.

One of the major challenges faced in tissue engineering is to create differentiated tissue of the appropriate size and shape. Tissue created without a functional vasculature is strictly limited in size by the constraints of oxygen diffusion; if the tissue is too large it will become necrotic before the host has time to create a new blood vessel supply. Thus there are many advantages in creating new tissue containing a functional vasculature. Additionally, as the new tissue may need to be produced at a site on the body remote from the defect, or on an immunosuppressed carrier animal or in vitro with an extracorporeal circulation, the blood supply for the new tissue must be defined, so that it can be brought with the tissue intact to the site of reconstruction.

The creation of skin flaps, a living composite of skin and its underlying fat, is a common technique used to repair tissue defects in reconstructive surgery. Because these flaps must retain their blood supply to remain viable after transplantation, the origin of the flaps is limited to those areas where there is an anatomically recognised blood vessel source. In order to overcome this limitation, skin flaps can be “pre-fabricated” by implanting short segments of blood vessels into a desired site, and utilising the resultant angiogenesis to vascularise a flap of the desired size and composition. Subsequently this vascularised flap can be transferred by microsurgery to the region of interest. This technique is, however, limited by the availability of donor tissue, and the disfigurement that results at the donor site.

In an extension to this technique, Erol and Spira (1980) demonstrated that the creation of an anastomosed arterio-venous (AV) loop beneath a skin graft could produce a vascularised skin flap.

However, while the generation of vascularised skin using an AV loop has been demonstrated, the production of other vascularised tissues suitable for grafting remains elusive. Vascularised adipose tissue, for example, is often demanded in reconstructive procedures; however, donor mature adipose tissue is extremely fragile, and will rapidly become necrotic if not immediately re-connected to a functional blood supply. Furthermore, the use of conventional autologous transplantation techniques involves “robbing Peter to pay Paul”, producing disfigurement at the donor site. The ability to produce new tissue with a defined vasculature would overcome this major shortcoming.

Khouri et al. (1993) and Tanaka et al. (1996) have demonstrated that an arteriovenous loop could intrinsically generate new, vascularised tissue when it was lifted from the body, sandwiched between sheets of collagenous matrix and isolated from the surrounding tissue within a plastic chamber. In the model described by Khouri et al., the generation of new tissue relied on the addition of recombinant BB-homodimer of Platelet-Derived Growth Factor (BB-PDGF), and even with this supplement the tissue was labile, peaking in volume at 15 days and subsiding by 30 days. Similarly, tissue growth in Tanaka's model, where the chamber was supplemented with β-Fibroblast Growth Factor (β-FGF or FGF-2), continued to increase in volume, peaking at 2 weeks, but returned to the levels of the unsupplemented control chambers after 4 weeks. This AV loop model is not generally known in th field of tissue engineering.

The classical notion that mature tissues do not contain stem cells has changed considerably in recent years. Many mature tissues which were previously regarded as largely non-self renewing are now considered to harbour a stem cell population. These stem cells possess the potential to change their phenotype in response to their environment, and may be able to provide a self-replenishing stem cell population (Prockop, 1997). Micro-environmental cues are considered to play a significant role in determining the behaviour of stem cells, for example, in initiating stem cell division and differentiation and/or maintaining stem cell quiescence. The cues and mechanisms behind these processes are far from being understood. However, it is clear that the ability to recruit, stimulate, proliferate and differentiate stem cells is the crux of tissue engineering. The behaviour of stem cells is largely studied in vitro, although a small number of in vivo studies have examined the behaviour of stem cells when injected either under the capsule of mature organs or systemically. These studies have a number of limitations in furthering the knowledge of the use of stem cells for tissue engineering. In particular, when the stem cells are injected into mature organs they must interact with an established micro-environment and derive a limited neovasculature from the host organ; when they are systemically injected they become widely dispersed. In order for stem cells to generate organs, it is expected that they will require an expandable vascular supply to accommodate and service de novo tissue generation. In order to assist in directing stem cell expansion, development and differentiation, an expandable microenvironment comprising an inert support and/or extracellular matrix is also expected to be required. We have now developed a model which satisfies these requirements, and holds great promise for the study of stem cells. Its application to tissue engineering is a significant advance in the state of the art.

It will be clearly understood that, although a number of prior art publications are referred to herein, this reference does not constitute an admission that any of these documents forms part of the common general knowledge in the art, in Australia or in any other country.

We have now developed a system for producing vascularised graft tissue, which is useful in transplant and reconstructive surgery, and also provides a useful model system.

### SUMMARY OF THE INVENTION

In a first aspect, the invention provides a method of producing donor vascularised tissue, suitable for transplantation into a recipient animal in need of such treatment, comprising the steps of:

a) creating a functional circulation on a vascular pedicle in a donor subject;

b) partially or totally enclosing the vascular pedicle within a fabricated chamber;

c) seeding the chamber with isolated cells or pieces of tissue;

d) implanting the chamber containing the vascular pedicle into a host animal at any site where such an anatomical construct can be created; and

e) leaving the chamber in the implantation site for a period sufficient to allow the growth of vascularised new tissue.

In one preferred embodiment, the method comprises the step after step (a) of surrounding the vascular pedicle with added extracellular matrix and/or a mechanical support. In another preferred embodiment, the method comprises a step after step (b) of adding growth factors, drugs, antibodies, inhibitors or other chemicals to the chamber.

Preferably in step (e) the chamber is left in the implantation site for at least 4 weeks, more preferably at least 6 weeks.

The vascularised tissue may be grown in vivo or in vitro, or may be in situ in the host.

More preferably the chamber is implanted in the donor body, beneath the skin, although it is not limited to subcutaneous insertion. While externalization of the chamber during tissue/organ growth is theoretically possible, the high risk of infection makes this a rarely used alternative.

For the purposes of this specification, the term “donor subject” is taken to mean an animal, especially a mammal and most especially a human, in which the donor vascularised tissue is created. For the purposes of this specification, the term “recipient animal” is taken to mean an animal, especially a mammal and most especially a human, that receives the donor vascularised tissue graft. It would be appreciated by those skilled in the art that as the generation of new vasculature, angiogenesis, in all warm blooded animals is associated with essentially the same physiological and pathological processes, methods disclosed herein are directly applicable to all warm blooded animals. The donor subject is preferably a mammal, and may be a human or a non-human animal. Preferred mammals include rodents, felines, canines, hoofed mammals such as horses, cows, sheep and goats, pigs, and primates. In a particularly preferred embodiment, the donor subject and recipient are human.

The person skilled in the art will appreciate that a “vascular pedicle” is an artificial or naturally occurring arrangement of blood vessels or vessel replacements that comprises an artery taking blood to the site of the construct and a vein carrying it away. Preferably the vascular pedicle comprises an arterio-venous (AV) loop or shunt. In an AV loop or shunt the artery is either joined directly to the vein or connected via a graft of a similar diameter so that there is no impediment to blood flow (for example as illustrated in FIG. 1). In one alternative arrangement, the artery and vein are both ligated and blood flow is via microscopic connections between the two (for example as illustrated in FIG. 3). In another alternative the artery and vein are in a “flow through” configuration with the blood vessels entering at one end of a semi-closed chamber and exiting at the opposite side (for example as illustrated in FIG. 4).

It would be appreciated by those skilled in the art that the term “functional circulation” as used herein describes a circulation that has at least one of the following properties: the vessels making up the circulation are patent, the vessels are capable of sustaining blood or blood-substitute flowing through them, the vessels are capable of supplying nutrients and/or oxygen to nearby tissue and the vessels are capable of forming new blood vessels by budding.

Optionally, the chamber may also be supplied with added extracellular matrix, for example matrix deposited by cells in situ, reconstituted basement membrane preparations such as Matrigel™ or laminin (mouse origin), Amgel™, Humatrix™, or laminin (all of human origin) with or without matrix metalloproteinase inhibitors, polylactic-polyglycolic acid variants (PLGA), fibrin or plasma glue (autologous or heterologous) with or without fibrinolysis inhibitors, or native collagen (autologous or heterologous) with or without collagenase inhibitors.

In a preferred embodiment, extracellular matrix-like polylactic-polyglycolic acid sponges, Dexon™ sponges, or sea sponges are added to the chamber. Combinations of matrices, such as PLGA sponges coated with one or more other matrix-forming components such as fibrin, laminin, fibronectin, collagen, low molecular weight hyaluronan and vitronectin are other preferred options. Freeze dried segments of tissues such as muscle or organs such as liver may be used as sources of matrix and growth factors. Preferably the segments of tissues or organs are taken from the same species as the donor subject, and most preferably taken from the donor individual.

In a particularly preferred embodiment of the invention, the donor subject is the same individual as the recipient animal, i.e. the graft is autologous. Alternatively the donor subject may be an immunocompromised animal, such as an athymic mouse or pig, and the recipient may then be a different individual, i.e. the graft is heterologous. Other permutations and combinations of these procedures may include the use of either autologous or immunocompromised blood vessels, cells, tissue segments or growth factors implanted back into either the original donor or a different recipient individual. Whether or not the “maturity” of the graft confers immunoprotection on a heterologous graft is another variant that can be tested using routine techniques.

The tissue or cells used in the chamber may be supplemented with additional growth factors selected from the group consisting of “homing” factors to attract stem cells from the circulation, exogenous grab factors such as α-Fibroblast Growth Factor (αFGF or αFGF-1), β-Fibroblast Growth Factor (βFGF-1 or βFGF-2), Platelet-Derived Growth Factor (PDGF), Vascular Endothelial Growth Factor (VEGF-A,B,C,D or E), Angiopoietin-1 and -2, insulin-like Growth Factor (IGF-1), Bone Morphogenic Protein (BMP-2 and -7), Transforming Growth Factor-α and -β (TGF-α, TGF-β), Epidermal Growth Factor (EGF), Connective Tissue Growth Factor (CTGF), Hepatocyte Growth Factor (EGF), Human Growth Hormone (UGH), Keratinocyte Growth Factor (KGF), Tumour Necrosis Factor-α (TNF-α), Leukemia Inhibitory Factor (LIF), Nerve Growth Factor (NGF), Granulocyte Macrophage Colony Stimulating Factor (GM-CSF) and other factors such as 3-isobutyl-1-methylxanthine (IBMX), insulin, indomethacin, dexamethasone, hyaluronan hexasaccharide, the PPAR-γ ligand Troglitazone, nitric oxide, prostaglandin E1, transferrin, selenium, parathyroid hormone (PTH), parathyroid hormone related peptide (PTrP), etc, many of which are promoters of angiogenesis or vasculogenesis. Antibodies, agonists or antagonists to some of these growth factors or inhibitors of the chemical mediators can also be used to influence the type of tissue formed and the rate of its formation. The person skilled in the art will readily be able to test which growth factor(s), anti-growth factor antibodies, or inhibitors, or combination thereof, are most suitable for any given situation.

The chamber may be used with autologous or heterologous cells, such as myoblasts transfected with Myo-D to promote formation of the skeletal muscle phenotype, stem cells with appropriate differentiation factors, keratinocytes seeded to produce thin skin constructs for face and neck reconstruction, etc. optionally the chamber may also comprise isografted or autologous cells selected from the group consisting of myoblasts, fibroblasts, pre-adipocytes and adipocytes, cardiomyocytes, keratinocytes, endothelial cells, smooth muscle cells, chondrocytes, pericytes, bone marrow-d rived stromal precursor cells, embryonic, mesenchymal or haematopoietic stem cells, Schwann cells and other cells of the peripheral and central nervous system, olfactory cells, hepatocytes and other liver cells, mesangial and other kidney cells, pancreatic islet β-cells and ductal cells, thyroid cells and cells of other ndocrine organs.

Alternatively the chamber may be used with additional autologous or isografted portions of skeletal or cardiac muscle, pancreas, liver, epididymal and other subcutaneous fat, nerves (peripheral, blood vessel-associated, etc), kidney, bowel, ovary, uterus, testis, olfactory tissue or glandular tissue from endocrine organs. For the purposes of the specification the term “pieces of tissue” shall be taken to encompass any aggregates of cells, with or without additional extracellular material such as extracellular matrix, either taken directly from an animal or produced as a result of manipulation of cells in tissue culture, or a combination of the two. In other variants such tissue segments may be rendered ischaemic, cell-depleted or necrotic in order to provide cues or signals to the surviving stem cells and other cells which may influence tissue development.

Depending on the nature of the supplementation provided to the cells, the vascularised tissue is enabled to differentiate in a particularly preferred embodiment, stem cells, together with appropriate extracellular matrix and growth factor supplements, are supplied to the chamber in order to produce vascularised, differentiated tissues or organs. Suitable pluripotent stem cells can be derived from:

a) blood;

b) bone marrow;

c) specific organs or tissues, including mesencymal stem cells;

d) cultured cells, which may be transfected or differentiated; or

e) placental stem cell banks.

To date we have used sourc s such as bone marrow, ischaemic skeletal muscle, and subcutaneous adipose tissue. Other potential sources of pluripotent stem c lls are blood, especially from a fetus or newborn individual but also from an adult, and human placenta. A number of stem cell banks such as bone marrow or cord blood banks are already established. Human embryos are a potential clinical source of stem cells, although legal and ethical issues precludes their use at present in some countries.

The type of differentiated cells produced depends on the origin of the stem cells, the local environment, the presence of tissue-specific growth or differentiation factors, and other factors. For example, unexpectedly we have observed that ischaemic skeletal muscle placed in the chamber with an AV loop differentiates into predominantly adipose tissue after 4-6 weeks. Without wishing to be limited by any proposed mechanism, we believe that in this case, mesenchymal stem cells in the muscle, together with the stimulus of acidic ischaemic metabolites, are potentially responsible for this differentiation. The chief advantage of using stem cells is their huge proliferative capacity, so that relatively few cells are required to generate a large colony for seeding the chamber and the AV loop.

Preferably the vascular pedicle, such as an AV loop comprises an artery joined to a venous graft, which is in turn joined to a vein. Alternatively the AV loop comprises an artery joined to a vein directly, or the AV loop comprises an artery joined sequentially to a venous graft, an arterial graft, and a vein. In another variant, which is useful where microsurgical anastomosis of vessels is technically difficult or impossible, a pedicle comprising the ligated stumps of an artery and vein (eg. the femoral vein) placed side by side in the chamber can be used as the blood vessel supply. In another preferred embodiment of the invention, the AV loop vessels flow in and out of the chamber from the same edge. In another variant the artery and vein are neither divided nor formed into a shunt, but instead flow in one side of the chamber and out the opposite side (see, for example, FIG. 4). In a third variant suitable for extremely small blood vessels, the artery and vein are divided and placed side by side in the chamber, the vessels both entering from the same edge; this is illustrated in FIG. 3.

The graft portion of the AV loop may be derived from the host or from a separate donor. Cold-stored or prefabricated vessels may also be used.

In one preferred embodiment of the invention, an additional step involves the incorporation of a nerve stump, so that tissue in the chamber may become innervated. Skeletal muscle, for example, requires proximity to a nerve for its maintenance and maturity; otherwise it will atrophy.

Preferably the chamber containing the vascular pedicle has a defined internal dimension. The internal dimensions, volume, and shape may be varied in order to influence the volume and shape of the new tissue being produced. For example:

a) the internal volume of the chamber may be increased, without altering the external size of the chamber, by providing thinner walls;

b) the shape of the chamber may be constructed to resemble that of the target organ or body part, such as an ear, nose, breast, pancreas, liver, kidney, finger or other joint;

c) the degree of permeability of the walls of the chamber may be varied; for example the chamber may include a semi-permeable membrane component to allow selective perfusion of molecules into and out of the chamber, or a plurality of perforations may be placed in the walls of the chamber to allow an increased flow of metabolites and metabolic by-products, growth factors and other factors that influence cell survival, growth and differentiation between the inside and outside of the chamber. The size, shape and number of the perforations may be selected according to the size of the donor vascularised tissue and the requirement to keep the contents of the chamber isolated from direct contact with the implantation site. Alternatively,

d) a semi-permeable component may be placed within the chamber in order to isolate “feeder” cells from immune reactions.

As an example of the latter, populations of fibroblasts r other cells can be transfected, then used as a source of the transfected gene product(s) within the chamber. This construct is placed within a semi-permeable pocket out of contact with the host's immune system. Drug delivery is used to switch the transfected gene on or off. These cells will survive by diffusion as long as they receive adequate nutrients, but will eventually die.

The surface chemistry of the chamber walls may be modified, in order to modify the interaction between the tissue and the chamber wall, to provide a stimulus for differentiation or to incorporate or be coated with a gel, such as alginate, which mediates the slow release of a chemical or biological agent to create a gradient.

The degree of internal support within the chamber may be varied, eg there may be:

a) no support;

b) a solid support which directs, encourages or inhibits the growth of the new tissue, or excludes new tissue, or is incorporated into the new tissue;

c) a transient support based on resorbable materials;

d) a porous supporting material which supports cell and vascular ingrowth, providing a skeleton over which the new tissue can be generated, eg sponge-like materials such as blown PTFE materials, PLGA sponges of variable composition and porosity, etc;

e) a support formed from materials which direct tissue differentiation, such as hydroxyapatite or demineralised, granulated bone.

Preferably the exterior surface of the chamber bears a means by which the chamber can be attached and/or immobilised to the desired region of the body.

In a second aspect, the invention provides a vascularised tissu graft, ie. the contents of the chamber, comprising differentiated tissue or an organ with a mature vascular supply.

Preferably the graft predominantly comprises tissue selected from the group consisting of adipose tissue, cartilage, bon , skeletal muscle, cardiac muscle, loose connective tissue, ligament, tendon, kidney, liver, neural tissue, bowel, endocrine and glandular tissue. More preferably the graft predominantly comprises vascularised adipose tissue, skeletal muscle, cartilage or bone tissue or tissue comprising pancreatic islet and/or ductal cells, kidney cells or liver cells.

In a third aspect, the invention provides a method of repairing a tissue deficit, comprising the step of implanting a tissue chamber according to the invention into a patient in need of such treatment, in which:

a) the tissue or “organ” graft is formed according to the methods of the invention, and;

b) retained for sufficient time to mature ie. to achieve the desired size, vascularity and degree of differentiation, and;

c) transferred to the desired recipient site; and

d) the blood vessels of the graft are microsurgically anastomosed to a local artery and vein.

For the purposes of the specification, the term “tissue deficit” will be taken to comprise a shortfall in the normal volume, structure or function of a tissue in the recipient. Such a tissue may be selected from, but is not limited to superficial tissues such as skin and/or underlying fat, muscle, cartilage, bone or other structural or supporting elements of the body, or all or part of an organ. The augmentation of otherwise normal tissues for cosmetic purposes, such as forms of breast augmentation, is also provided by the invention. A person skilled in the art will readily recognise that such a tissue deficit may be a result of trauma, surgical or other therapeutic intervention, or may be congenitally acquired.

In a fourth aspect, the invention provides a method of providing a subject with a gene product, comprising the steps of:

a) constructing a tissue chamber according to the invention to create vascularised tissue from a patient in need of such therapy;

b) removing the chamb r with its vascularised tissue and culturing the chamber assembly in vitro;

c) transforming cells of the tissue in the chamber with a desired gene; and

d) implanting the chamber or the contents minus its chamber into the patient.

The timing of the genetic transformation of the tissue-producing cells can be varied to suit the circumstances, for example the cells may be transformed at the time of setting up the chamber construct, during the incubation, or immediately prior to transplantation.

The provision of gene products can take several forms. One example is the transfection of myoblasts with the Myo-D gene to create tissue with a normal skeletal muscle phenotype. Such transfected cells may then be seeded into the desired chamber, matrix and AV loop to generate vascularised skeletal muscle. This may have implications for the treatment of muscular dystrophy and other genetically inherited muscle diseases. A second example is the transfection of pancreatic islet cells with a “healthy” phenotype and their seeding into the chamber. This approach may prove to be useful in the treatment of diabetic patients. In a third example, cells are transfected with a growth factor gene or an angiogenesis-promoting gene, such as PDGF, bFGF or VEGF, prior to seeding them into the chamber together with the AV loop and selected matrix. This continuous production of growth factor is designed to speed up the rate of development of, and the rate of new blood vessel formation within, the new tissue/organ.

In a fifth aspect, the invention provides a model system for vascularised tissue, comprising a tissue chamber containing a vascular pedicle of the invention and optionally an extracellular matrix, operably connected to an extracorporeal circulation apparatus and renal dialysis filter. The extracorporeal circulation apparatus and renal dialysis filter may be of any suitable conventional type. The cells forming the tissue in the chamber are optionally transformed so as to express a heterologous gene. This model system may be used for culturing, recruiting, growing and studying the behaviour of stem cells or tissue containing precursor cells, either in vitro or in vivo. Because of the ability to alter the environment of the chamber with added growth, differentiation and chemical factors, it is possible to produce a wide variety of tissues and organs by this process.

The ability to generate autologous vascularised tissue of a defined composition and at any anatomical site in the body where it is possible to create an arterio-venous loop or suitable vascular pedicle has many other applications. At its localised site the tissue in the chamber may, for example, be manipulated by

a) gene transfection,

b) administration a local drug or other “factor”, or

c) creating a site of circulatory stem cell homing.

Furthermore, the tissue and exudate in the chamber may readily be harvested to monitor progress of tissue growth and development. Above all, it is the ability to grow and transplant new vascularised, differentiated tissues or organoids that sets this invention apart from others.

For the purposes of this specification it will be clearly understood that the word “comprising” means “including but not limited to”, and that the word “comprises” has a corresponding meaning.

### BRIEF DESCRIPTION OF THE FIGURES

FIG. 1 illustrates how the femoral artery and vein are anastomosed microsurgically to a vein graft of similar diameter to form a loop (shunt). The AS loop is placed as shown in a plastic chamber (made of polycarbonate or poly-L-lactic acid, etc), the lid secured, and the chamber optionally filled with an extracellular matrix with or without added cells or growth factors. The chamber is anchored in position relative to the surrounding tissue by means of stay sutures through external holes.

FIG. 2 shows a configuration similar to FIG. 1, except that the lid of the chamber is dome-shaped and the edges of the chamber are more rounded to minimise wound breakdown.

FIG. 3 depicts an example of the thin-walled chamber used for the pedicle model. In this case an artery and a vein are ligated distally and placed adjacent to each other. Microscopic connections between the artery and vein become established, and form an AV loop in a similar manner to that shown in FIGS. 1 and 2.

FIG. 4 shows a model chamber similar to that in FIG. 3, but with exit holes for the blood vessels at either end of the chamber. This allows an undivided, dissected length of blood vessels, placed side by side, and in some variants surrounded with extracellular matrix, to form new tissue.

FIG. 5 shows the inner aspect of an AV loop-containing chamber, 7 days after insertion. Fluorescence microscopy shows labelled fibroblasts evenly distributed across the chamber surface, magnification×160 (see Example 2).

FIG. 6 shows a reconstructed “breast” on a male rabbit, constructed using a vascularised, tissue-engineered fat and connective tissue flap created at a remote site (the groin region) in the same rabbit (see Example 10).

### DETAILED DESCRIPTION OF THE INVENTION

The invention will now be described in detail by way of reference only to the following non-limiting examples and drawings.

Experimental Procedures

Preparation of Tissue Chamber

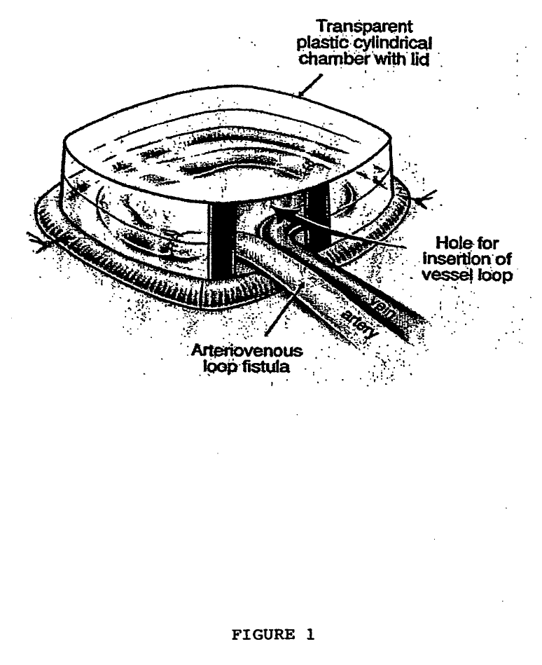

A custom-made polycarbonate chamber was pr pared. It has a top and a bottom, and when the two halves are sealed together th internal volume is 0.45-0.50 ml. The general construction of the chamber is illustrated in FIG. 1.

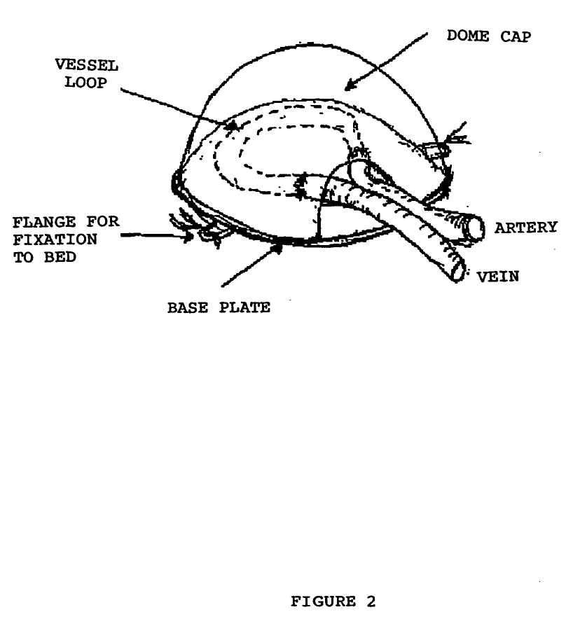

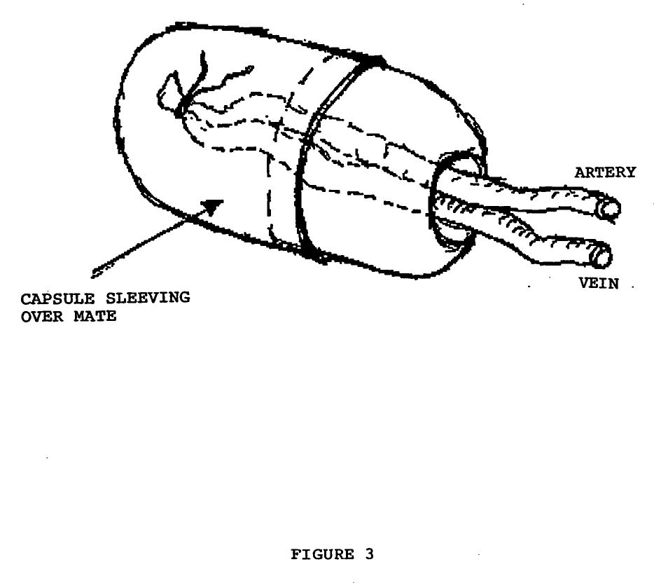

The basic chamber for use in rats is made of polycarbonate. In one variant the chamber is made of polylactic acid or PLGA. The chamber is in the shape of a cylinder of external dimensions 14 mm diameter and 4 mm high, with a saw cut on one side to create an opening for the blood vessel entry and exit. Another variant has cut openings on opposite sides of the chamber to allow blood vessels to flow in one side and out the other. The chamber has a base and a removable lid. The base has holes to allow anchoring of the chamber to subcutaneous tissue. The internal volume is approximately 0.45-0.50 ml. The internal volume of this basic chamber can be varied, maintaining the same external volume, by using thinner walls, which may even be as thin as a standard plastic film used in food storage. An alternative design is in the shape of a “dome” with more rounded edges, as shown in FIG. 2. Other variants include an elongated, flattened cigar shape as shown in FIG. 3 which fits readily into the subcutaneous space in the groin. For the purposes of specific grafts, the shape of the chamber may be designed to mimic the shape or contours of a particular body part, for example a human finger joint or thumb, human ear, human nose, human breast, etc.

The size of the chamber can be scaled up or down to suit the size of the host. Hence the internal volume for a chamber to be used in a mouse may be approximately 0.1-0.2 ml, in a rabbit 10-12 ml, but in a human can be up to approximately 100-200 ml.

The chamber may optionally be sealed. In the standard version the opening allows limited contact with the surrounding tissue and total uninterrupted contact with the blood supply. In a sealed variant, the opening is engineered to allow just enough space for the ingoing artery and outflowing vein without crushing the blood vessels. The vessel ports are sealed, for example with fibrin glue, to avoid contact of the developing graft with sounding tissue.

The surface of the polycarbonate chamber can be left in its native hydrophobic state, or can be rendered relatively more hydrophilic by the use of polylactic acid or the pre-treatment of polycarbonate with a thin film of poly-L-lysine. In one useful configuration, the surface of the chamber comprises a plurality of perforations, allowing increased contact with growth factors in the surrounding tissue. The size and shape of the perforations may be tailored to optimise the passage of the desired factors, while minimizing or preventing the passage of cells.

If the chambers are made of glass or Pyrex they can be coated with silicone.

The chamber design should ideally fit comfortably into the recipient site, and should be of a rounded shape and of a sufficiently small size to avoid wound break down.

The internal contents of the chamber are sufficiently large to accommodate an osmotic pump (eg. an Alzet™ osmotic mini pump) to deliver drugs, growth factors, antibodies, inhibitors or other chemicals at a controlled rate. In one alternative method of drug/factor delivery, the osmotic pump may be placed subcutaneously outside the chamber with a plastic tube leading from the pump placed inside the chamber, eg. at the centre of the AV loop.

Creation of an Arteriovenous (AV) Shunt Loop Inside the Tissue Chamber

The basic model has been described by Tanaka et al (1996). Briefly, male Sprague-Dawley rats (225-285 g) were anaesthetised with intraperitoneal phenobarbitone (50 mg/kg; 2.5 ml of a 6 mg/ml solution). Under sterile conditions an inferior-based flap was created in the right groin to expose the femoral vessels from the inguinal ligament to the superficial epigastric branch. A longitudinal incision was made in the left groin to harvest the left femoral vein from inguinal ligament to the superficial epigastric branch. This vein graft (approximately 1.5-3 cm long; usually 2 cm) was interposed between the recipient right femoral vein and artery at the level of the superficial epigastric artery by microsurgical techniques using 10-0 sutures. The shunt was placed into the chamber, the lid closed and the construct sutured to the groin musculature with the aid of small holes on th base of the chamber. An adipose layer was placed over the chamb r and the wound closed with 4-0 silk sutures.

The growth chambers with the AV shunts were harvested at either 2, 4 or 12 weeks post implantation.

Assessment of Vascularisation and Tissue Creation

At the specified time of exploration, the chamber was opened, and the vessels cleaned and tested for patency. The vessels were tied off with a 5-0 silk suture at the entrance of the chamber and the flap harvested. In 2 of the 5 rats in each group the flap was perfused, via the aorta, with India ink prior to harvest (details below). The flaps were assessed for volume and weight and placed in buffered 10% formal saline (BFS) for histological examination. The animals were sacrificed with an intracardiac dose of sodium pentabarbitone (˜3 ml of 250 mg/ml solution) at the completion of the exploration.

Tissue Mass and Volume

The tissue in the chamber was removed and its wet weight and volume recorded. The volume of the tissue was assessed by a standard water displacement technique. The tissue was suspended by a 5-0 silk suture in a container of normal saline which had been zeroed previously on a digital balance. Care was taken not to touch the container with the specimen. The weight recorded was the volume of the tissue specimen (with a density equal to that of normal saline, 1.00 g/ml). The mass of the specimen was assessed at the same time on the same digital scale by allowing the tissue to rest on the base of the container, and recording the weight.

India Ink Perfusion

In order to perfuse the flaps with India ink, the abdomen was opened via a midline incision. The intestines were gently retracted to the periphery and the periaortic fat stripped away. The proximal aorta and inferior vena cava were ligated. The aorta was cannulated with a 22-gauge angiocatheter which was secured with a distal suture around the angiocatheter and aorta. A venotomy was carried out in the inferior vena cava. The aorta was perfused with 10 ml of heparinised saline to flush out the retained blood, the animal was sacrificed with intracardiac sodium pentabarbitone (3 ml of a 250 mg/ml solution), the aorta infused with 3 ml buffered 10% formol saline (BFS) and then with 5 ml India ink in 10% gelatin. The flap vessels were then tied off. Tissue from the chamber was removed, fixed in BFS, cleared in cedar wood oil and the pattern of vessels visualised microscopically using transmitted light and image analysis (Video Pro™ imaging).

Histology

Specimens were fixed in buffered formol saline and embedded in paraffin. Sections (5 μm) were cut and stained with either haematoxylin & eosin (H & E) or Masson's Trichrome.

### EXAMPLE 1

### Creation of Vascularised Tissue in Chambers with an AV Loop

Three groups of five rats each were used. Each group had an identical procedure performed as described above, and the growth chambers with the AV shunts were harvested at either 2, 4 or 12 weeks post implantation.

The average mass of the AV shunt vessels prior to insertion was 0.020 g (exsanguinated) and 0.039 g (when full of blood). Two weeks after insertion the AV shunt and its surrounding tissue weighed 0.18±0.03 g. The mass increased progressively being 0.24±0.04 g at 4 weeks and 0.28±0.04 g at 12 weeks. The volume of the new tissue closely paralleled its weight. The increase in weight but not volume between 2 and 12 weeks was statistically significant (P<0.05, ANOVA/Dunnett's test).

Two weeks after implantation the AV loop was surrounded by a mass of coagulated exudate containing varying amounts of clotted blood. At 4 weeks the mass of tissue around the loop was larger and firmer, especially in its central part. By 12 weeks the newly formed tissue surrounding the loop had increased still further in volume and now filled approximately two-thirds of the chamber. The surface coagulum was no longer visible, and the whole mass had a uniformly firm consistency.

After 2 weeks of incubation the AV shunt was surrounded by a cuff of newly-formed connective tissue composed of fibroblasts, thin collagen fibres and vascular sprouts, arranged roughly vertical to the shunt. Inflammatory cells, both neutrophils and macrophages, were present in moderate numbers in the outer part of the newly formed tissue and in the surrounding mass of coagulated inflammatory exudate. In occasional sections, branches of newly-formed blood vessels arising from the venous lumen of the AV shunt could be identified.

In the 4 weeks incubation group, the newly formed tissue was more mature. The zone closest to the AVS contained a dense plexus of newly formed vessels embedded in mature collagenous stroma. Outside this layer was a less mature zone similar to the newly formed tissue in the 2 weeks specimens. Most of the surrounding coagulum was no longer visible, and only small numbers of inflammatory cells were present in the newly formed tissue. As at 2 weeks, communications between the AV shunt and the newly formed vessels were visible in some sections.

Twelve weeks after incubation, the newly formed tissue had matured still further, and consisted of dense collagenous connective tissue with fibroblasts aligned parallel to the outer margin of the AV shunt. There was no apparent decrease in vascularity and newly formed vessels formed a dense plexus throughout the connective tissue. Few inflammatory cells were visible.

At all three time points, the specimens which were injected with India ink gave a clearer picture of the extent and density of the newly formed vasculature. In most specimens almost all vessels contained carbon in their lumen, indicating that they communicated with the AV shunt.

Ideally, newly formed tissue must be stable and capable of retaining its shape. The tissue formed around an AV loop has both these characteristics. At 2 weeks the mass within the chamber is soft and readily deformed. By 4 weeks it is firmer and more rigid, and at 12 weeks it has the physical characteristics of mature connective tissue. Surprisingly, growth is continuous for at least 12 weeks after implantation, with no indication of resorption or regression of the newly formed tissue with increasing maturity.

### EXAMPLE 2

### Chambers with Rat Dermal Fibroblasts

Culture of Rat Dermal Fibroblasts

Rat skin was harvested in a 6 cm by 4 cm ellipse from the groin area of an inbred Sprague-Dawley rat line (Monash University Animal Services, Clayton, Victoria, Australia). The inbred line comprised animals resulting from at least 20 generations of brother-sister matings.

The epidermis was trimmed off. Segments of dermis were cut into 2 m=by 2 mm squares and 10 pieces were placed onto a sterile Petri dish and attached to the base using rat plasma “glue”. This glue was made by the addition of 2 ml of rat plasma, prepared from Sprague Dawley rats, to 0.3 ml of 2% calcium chloride. The glue was allowed to set for 10 min at 37° C. Complete culture medium, comprising Dulbecco's Modification of Eagle's Medium (DMEM), 10% fetal calf serum, penicillin and streptomycin and glutamine, was added to the culture dish. The skin segments were left undisturbed for 7 days, then the medium was changed. There was considerable outgrowth of fibroblasts by 10 days, at which time the skin segments were removed. The fibroblasts were subcultured twice at weekly intervals, each time growing the cells in 75 cm² and 175 cm² culture flasks respectively.

The fibroblasts were labelled with two fluorescent labels, bisbenzamide (EB) and carboxyfluorescein diacetate (CFDA). Three ml of 0.1% trypsin in phosphate buffered saline (PBS) at pH 7.4 was added to a 175 cm² cell culture flask containing confluent fibroblasts for 5 min at 37° C. The trypsin was neutralized by the addition of 17 ml of complete DMEM media. The cell suspension was centrifuged at 2000×g for 10 min. The cell pellet was resuspended in 3 ml of media and the suspension transferred in three 1 ml aliquots to Eppendorf tubes. To each Eppendorf tube 13.5 μl of a 10% CFDA solution and 20 μl of BS were added. The tubes were incubated for 1 h at 37° C. and shaken gently every 15 minutes. The cells then were transferred into a 175 cm² flask and recultured. CFDA persists in the cytoplasm of cultured cells and survives the division of cells into daughter cells. CFDA fluoresces maxmimally at 513 nm; BB fluoresces maximally at >430 nm. Labelled cells were protected from light, in an effort to maintain maximal fluorescence.

Cell Counting

Prior to the addition of cells to the chambers, the fibroblast culture flasks were trypsinized and the trypsin neutralized. 10 μl of suspended cells were counted using a hemocytometer, and 0.05% Evan's blue dye in a 1:10 ratio. The solution was centrifuged and the resulting cell pellet suspended in an appropriate volume of bovine collagen solution to yield a cell concentration of 1 million cells/ml.

Rat Tail Tendon Collagen (RTTC)

The tendons from six rat tails were harvested and diced into 2×2×2 mm cubes (yield approximately 10 g). Four hundred ml of cold 0.5 M acetic acid was added and the mixture homogenized and left stirring at 4° C. for 24 h. The homgenate was centrifuged (3000 rpm×20 min) and the supernatant harvested. This extraction procedure was repeated twice with further additions of 300 ml of cold 0.5 M acetic acid. To the pooled extracts a solution of 5 N NaCl was added slowly, with magnetic stirring at 4° C., until the final concentration of salt was approximately 0.7 M (100 ml of 5M NaCl added to every 600 ml of extract). The solution was left for 1 h to allow full precipitation of the native collagen. The precipitate was collected by centrifugation (3000 rpm×20 min at 4° C.), redissolved in 200 ml of 0.5 M acetic acid and dialysed twice against 2 l of cold 0.5 N acetic acid for 24 h, and twice against sterile, cold distilled water, the final dialysis solution containing a few drops of chloroform on the surface. This results in a sterile stock solution of RTTC of approximately 3 mg/ml, the concentration checked by a Bradford protein assay (Bio Rad) with a Type I collagen standard.

Preparation of Chambers

All procedures were carried out in a cell culture hood using st rile technique. Chambers were coated internally with RTTC by addition of 200 μl of 2.5 mg/ml RTTC solution, pH 7.4, to each half chamber. Chambers were incubated for 1 h at 37° C. to allow gel formation and dried for 24 h. After rinsing with PBS to remove residual salt crystals, 0.25×10⁶ of fluor scently labelled fibroblasts in 150 μl of complete MM were added to each half chamber. After allowing 1 h for adherence of the cells, chambers were immersed in complete DMEM and incubated at 37° C. under 5% CO₂ in air for 24 h. The density of labelled of cells was determined by counting the number of cells in 7 randomly selected fields of each half chamber using a ×10 objective.

Insertion of Chambers

Two groups of 6 inbred male Sprague-Dawley rats, weighing between 230-280 g, were used. Two chambers were inserted into the inguinal region of each rat, the chamber in the right side containing an AV shunt (prepared as described above) and that in the left side containing no shunt. In 6 rats chambers were removed 2 days after implantation. The remaining 6 chambers were removed 7 days after implantation.

Examination of Chambers After Removal

The chamber was removed, the AV shunt examined for patency and the flap removed. Ten μl of 0.05% Evan's blue dye was added to each half chamber and incubated for 5 min at 37° C. The base of each half of the chamber was then examined, using a ×10 ocular, to determine the number of Evan's blue-stained and fluorescent cells in 7 randomly selected microscopic fields. The number of labelled cells in 7 random fields on the surface of the AV shunt was then determined.

Two days after insertion the shunt and surrounding tissue covered approximately 20% of the surface of the chamber; by 7 days this had increased to approximately 30%. On this basis the overall density of cells in the chamber containing an AV shunt was calculated by summation of the density of cells on the surface of the chamber and 20% (2 days) or 30% (7 days) of the labelled cells on the surface of the AV shunt.

Paired t-tests were used to compare number of cells per grid in the control and experimental chambers and the preop rative number of cells per grid using Microsoft Excel™ and Graph Pad Prisms software (San Diego, Calif., USA).

After counting, the shunt and surrounding tissue was fixed in 10% formol saline, embedded in methacrylate and thin sections prepared and stained with either haematoxylin and eosin or Masson's trichrome.

Comparison Between Labelling with Bisbenzamide (BB) and Carboxyfluorescein Diacetate (CFDA)

In both in vitro cultures and the in vivo chambers the number and distribution of labelled cells at the two wavelengths examined (430 nm for BB; 573 nm for CFDA) was the same. No cells were identified as being labelled with only one fluorescent dye. Hence in the results which follow “fluorescent cells” refers to cells labelled with both BB and CFDA.

Macroscopic Findings

The AV loop was patent in every chamber.

Two days after insertion the AV shunt covered approximately 20% of the surface of the chamber. By 7 days the area covered by the AV shunt and new tissue arising from it had increased to approximately 30%.

The 2 day mean weight of the shunt was 0.12±0.017 g and the mean volume was 0.12±0.014 ml. By 7 days the mean weight had risen to 0.23±0.018 g and the mean volume to 0.21±0.015 ml.

Density of the Labelled Cells

The density of the labelled cells in empty and AV shunt containing chambers is shown in Table 1.

TABLE 1
Density of labelled cells. (mean number/grid) in empty
and AV shunt containing chambers, pre-operatively and 2 and 7
days after insertion.

| | | | AV Shunt-containing | |
|---|---|---|---|---|
| Time after | Pre- | Empty | chamber | |
| Insertion | operative | Chamber | In chamber | Total* |
| 2 days | 8.6 ± 1.74 | 4.0 ± 0.94 | 4.8 ± 0.59 | 5.7 ± 0.62 |
| 7 days | 10.2 ± 1.7 | 4.8 ± 1.3 | 11.7 ± 1.4 | 15.5 ± 1.1· |

*Total density calculat d as number of cells/grid on chamber surface plus 20% (2 days) or 30% (7 days) of labelled cells in surface of tissue surrounding the AV shunt.
#Increase above density in 7 day empty chambers is significant (p = 0.011).

It can be seen that in all chambers the cell density decreased in the early stages after implantation, the values in all 2 day chambers being less than their pre-insertion density. Two days after insertion there was no significant difference in the density of cells in empty and AV shunt-containing chambers.

At 7 days the density of the cells in empty chambers did not differ significantly from the density 2 days after insertion. In contrast, the cell density in AV shunt containing chambers increased to almost 3 times its 2 day value, and both the density of cells in the grid and the density (after allowing for the number of labelled cells in the tissue surrounding the shunt) were significantly greater than the density in empty chambers (p=0.013).

Evan's blue staining showed that in all chambers examined virtually all labelled fibroblasts were viable, with less than 1% of cells taking up the Evan's blue dye.

Histological Findings

After 2 days incubation the vessels of the AV shunt were surrounded by blood clot and coagulated inflammatory exudate. Small numbers of fibroblasts were visible migrating from the vascular adventitia into coagulum.

By 7 days, many more fibroblasts were present within the coagulum, and early vascular sprouts were visible arising from the outer aspect of the AV shunt.

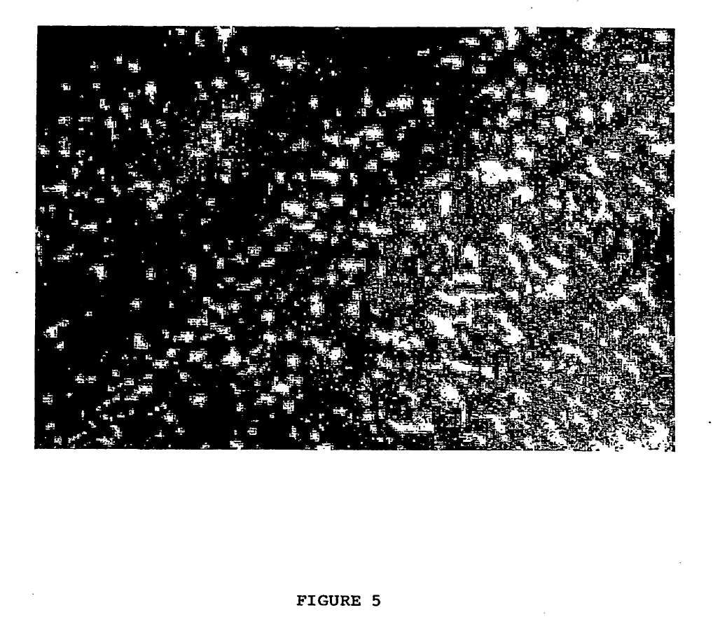

At both 2 and 7 days fluorescent studies showed labelled fibroblasts on the surface of the coagulum surrounding the AV shunt, but labelled cells were not seen within its substance. The inner aspect of an AV shunt-containing chamber removed 7 days after insertion is shown in FIG. 5.

### EXAMPLE 3

### Differentiation of Stem Cells in Implanted Tissue Chambers

Skeletal muscle, pancreas, fat, liver and kidney were aseptically removed from four inbred Sprague-Dawley rats. They were chopped into 1 mm cubes and placed in a tissue culture-grade petri-dish (15-20 pieces each 7 cm² of culture surface) containing 1-2 ml of complete serum-free DMEM. They were then incubated for a minimum of 24 h and up to 3 days. At the appropriate time 4-6 pieces of tissue were adhered in a plasma clot to each side of a chamber of the type described in Example 2. The chamber was then seeded with the AV loop and closed. The proximal end of a femoral nerve was placed inside one half of the chambers containing skeletal muscle explants. After 4-6 weeks the rats were sacrificed and the chambers examined.

After 4-6 weeks, the contents of chambers with tissue explants differed from the contents of chambers without tissue explants, in that they contained new and different cell phenotypes. In all cases most of the necrotic tissue explants had been replaced by clumps of new cells.

In the most dramatic of these experiments, 8 of the 11 chambers seeded with skeletal muscle explants contained up to two thirds of their volume with mature, well-vascularised adipose tissue together with mature skeletal muscle fibres, surrounded by a thin capsule. The mature region of the new tissue contained up to 90% vascularised adipose tissue. The remaining chambers also had a lesser proportion of mature adipose tissue and skeletal muscle fibres.

The chambers seeded with portions of pancreatic tissue had a large population of well-demarcated large ovoid eosinophilic cells, many giant cells and other smaller cells.

Without wishing to be limited by any proposed mechanism, we believe that a “stem cell” population, either attracted into the chamber from a circulating stem cell source by the necrotic tissue explants, or contained within the tissue explants, has given rise to the new tissue. In either case a very small amount of explant tissue was used, in comparison to the large amount required to isolate stem cells, and our results indicate that this is a novel and efficient method to obtain stem cells. The stem cells may have differed with respect to their degree of commitment to a particular tissue type, or else they may have responded to cues expressed by the unique microenvironment of the different explants, to proliferate and differentiate into the different cell types observed.

The generation of encapsulated adipose tissue described here is, to our knowledge the first time that such a neo-organoid has been grown de novo on its own artery and vein.

A detailed study of the spatio-temporal and dynamic changes in the chamber and the mechanism by which these events give rise to the neo-organ may also have applications in defining in vivo stem cell availability and behavior. The chamber model is superior to any other in vivo model available so far, since it enables a wide variety of manipulations of the chamber contents and environment and stem cell sources. Furthermore, it enables a study of stem cells in a naive environment without the influences of other nearby tissues, as opposed to the growth of stem cells in an established tissue.

The finding that muscle explants can result in the generation of a neo-organ, consisting almost entirely of mature adipose tissue, indicates that:

a) a stem cell population can successfully seed the chamber;

b) the chamber model supports the plasticity of stem cells;

c) a satisfactory, appropriate and adequate neovascularisation develops with, integrates and supports the tissue construct;

d) the constructs are not overcome by fibroblastic in-growth; and

e) the constructs are not overcome by inflammatory cells.

These results demonstrate that application of the chamber model to tissue engineering is feasible, and represent a significant advance in the art of “tissue engineering”.

### EXAMPLE 4

### Effect of Matrigel

A pilot study was devised to determine if there was any initial loss of Matrigel during 20 minutes of contact with the AV loop. Based on the results of the pilot study, time periods of 2, 4 and 8 weeks were chosen. At the 4 week time period a further comparison was done with growth factor-reduced Matrigel. Six male Sprague-Dawley rats were used per group, each weighing between 220 and 280 g. The arterio-venous loop procedure was carried out as described in the Experimental Procedures.

Matrigel (Collaborative Research Inc, Bedford, Mass., USA) was divided into in sterile 10 ml aliquots at an approximate concentration of 12 mg/ml in DMEM containing 10 μg/ml of Gentamycin (Becton Dickinson). The Matrigel was stored at −20° C. and prior to use was thawed overnight at 4° C. Throughout the preparation process the Matrigel was kept on ice and manipulated using pre-cooled pipettes. Growth factor reduced (GFR) Matrigel was prepared from matrigel essentially as described by Vikicevic et al (1992). This involved an additional fractional ammonium sulphate step. The protein concentration of the resultant GFR Matrigel was verified by Bradford protein assay and by Coomassie blue staining after SDS-PAGE to be consistent with that of normal growth factor-replete Matrigel.

Under sterile conditions, 0.5 ml of Matrigel was added to each sterile chamber at room temperature where it gelled rapidly (within 15 seconds). The chamber with matrigel was then placed in position in the rat's right groin. The Matrigel is gelatinous at room temperature, enabling immersion of the loop within it. In the pilot study the AV loop was made and immersed in the Matrigel for 20 minutes before implantation, to d termine whether there was any initial loss of Matrigel from the chamber due to liquefaction of the matrix.

For the time course studies the new tissue flaps were harvested at 2, 4 and 8 week periods. The flaps were harvested at the above time periods, and assessed for weight, volume and histology. Statistical analysis was carried out comparing the 2, 4 and 8 week groups with each other and the AV loop alone (See Example 1). A further comparison was done at 4 weeks between Matrigel, GFR Matrigel and the AV loop alone at 4 weeks.

In the pilot study Matrigel proved easy to manipulate in vitro. There was minimal loss of Matrigel after 20 minutes of contact with the AV loop.

In an AV loop alone (no added matrix), the average weight of the new tissue flap formed after 4 weeks was 0.24±0.04 g, and the average volume was 0.23±0.03 ml. These results acted as the control for this experiment and Example 5.

At two weeks the average weight of flap in chamber supplemented with Matrigel was 0.32±0.03 g and volume was 0.30±0.03 ml. This was significantly greater then the 4 week loop alone flap (p=0.05). At four weeks the flaps were slightly heavier than the 2 week flaps, with an average weight of 0.35±0.03 g and a volume of 0.33±0.03 ml. A comparison of these two groups showed no statistical significance. The weight (p=0.01) and volume (p=0.01) were both significantly greater than the control flaps produced by loop alone.

At 8 weeks the flaps had regressed, with an average weight of 0.18 g±0.02 g and volume of 0.16 ml±0.02 ml. Statistical analysis reveals that this is highly significant in weight (p=0.002) and volume (p=0.001) when compared with both the two week flaps and the four week flaps weight (p=0.0005) and volume (p=0.0003). For this longer time course 8 rats were operated on to compensate for infection or dehiscence. No such problems were encountered, so all 8 have been included in the analysis.

The GFR Matrigel flaps were smaller than the normal Matrigel flaps at 4 weeks, weighing on average 0.27±0.02 g. A comparison of weights showed no statistical significance. The volume was 0.24±0.01 ml; this was significantly less than the normal Matrigel (p=0.04). The GFR flaps were still larger than the loop alone at the same time period (not statistically significant). One of the chambers became infected, and had to be removed. As a consequence there were 5 animals examined in this group.

At 2 and 4 weeks a significant flap of tissue had formed when compared to chambers containing the loop alone at day 0. There was residual Matrigel in the chamber, and strands of microvessels were visible running from the flap edge into the Matrigel. Microfil injection demonstrated good filling of flap vessels, including the advancing microvessels. This appearance was not apparent at 8 weeks, when the flaps were smaller and with a more regular smooth surface. At 8 weeks there was only residual fluid in the chamber, and no viscous Matrigel was visible.

Histological examination showed that at 2 weeks there were many immature vessels extending to the flap edge, with haemorrhage within the peripheral tissue. There was early collagen formation in the central portion and areas of unincorporated Matrigel within the flap.

At 4 weeks the vessels had matured into arterioles and venules, with larger branching vessels arising from the loop and smaller branches at the periphery. There was still some unincorporated Matrigel and small amounts of haemorrhage. The unincorporated Matrigel contained sparse fibroblasts and the occasional vessel. The general impression was of a maturing but still growing flap with good vessel formation.

At 8 weeks the flap tissue appeared more mature, with denser collagen and larger vessels nearer the loop. It was less cellular with less vessels. A capsule had started to form around the generated tissue, and there was residual Matrigel remaining within the flap.

The GFR Matrigel flaps appeared to be more mature, with larger vessels in the centre and less active angiogenesis at the periphery. There was evidence of early capsule formation and in some specimens more inflammatory cells were present.

At all time courses Microfil injection demonstrated good vascular connection between the loop and the flap vessels.

### EXAMPLE 5

### Effect of Poly-L-lactic Polyglycolic Acid (PLGA)

(a) Ply Prepared by the Salt-Leached Method.

A PLGA insert for the tissue chamber was constructed using a particulate leaching method as described by Patrick et al (1999). In essence PLGA is dissolved in chloroform and mixed with NaCl. After evaporation of the chloroform the resulting scaffold is machined to the desired shape. The salt was then leached from it leaving interconnected pores. The pore size is a reflection of the size of the salt particle used. In this experiment pores of 300-400 μm and a porosity of 84% were made. The PLGA was machined in two parts so as to fit inside the polycarbonate chamber. The lower part comprised a base plate containing a groove for the loop and the upper part comprised a flat disc to cover the loop and base plate. The PLGA discs were 1.4 mm in diameter by 2.5 mm thick. The PLGA was sterilised and pre-wetted by soaking in 100% alcohol for 30 minutes on a mechanical stirrer then subjecting them to three 30 minute washes in sterile saline washes, also on a mechanical stirrer.

The arteriovenous loop was prepared as described above, and placed into the base plate of PLGA sitting in the chamber. The superior disc was placed on top and the chamber closed. Each group of rats contained 6 male Sprague-Dawley rats, with each rat weighing between 220 and 280 grams. The chambers were harvested at either 2 or 4 weeks. Weight, volume and histology were assessed at both time periods. Immunohistochemical staining of flap sections for α-actin was carried out to detect myofibroblasts. In each group, one chamber was excluded, one due to infection and the other to dehiscence, leaving 5 rats in each group.

At 2 weeks the vessels had almost entirely vascularised the construct, with some uninvolved PLGA at the tip. The capsule had begun to form proximally near the portal. At 4 weeks the construct was entirely encapsulated, and had shrunk and retracted, withdrawing from the sides of the chamber. Micro-fill injection demonstrated the extent of vessel penetration.

The 2 week flap weight was 0.43±0.05 g and the volume 0.38±0.04 ml. The 4 week flap weight was 0.33 g±0.04 g and the volume 0.29 ml±0.04 ml. A comparison between the 2 and 4 week groups showed a reduction in flap size between 2 and 4 weeks. This result was not statistically significant. Further comparison with other experiments was not possible due to the presence of PLGA retained within the flap, which skewed the results.

At both 2 and 4 weeks there was extensive vessel outgrowth, with branching vessels found up to the edge of the PLGA. Arterioles had formed, and healthy branching angiogenesis was seen coming from the loop. The cellular infiltrate was lying on the matrix and on the surface of the structure. A capsule had formed on the proximal part of the flap only at 2 weeks. α-Actin stain showed that this capsule contained myofibroblasts. At 4 weeks the capsule was thicker proximally, with more myofibroblasts and had extended to encompass the whole flap.

(b) PLGA Prepared by a Fiber-Spun Method.

The vascular loop model described in Example 1 was used in this experiment. The AV loop was placed within a round polycarbonate chamber (0.5 ml volume) filled with a PLGA disc (75% poly-L-lactic acid/25% polyglycolic acid) as the scaffold. The PLGA scaffold was either manufactured by the salt leaching method described above or a fiber spun technique. Each group comprised five animals. After 4 weeks incubation and immediately before harvest heparinised India Ink was infused i.v. for 5 min. Tissue from the chamber was harvested, fixed in buffered 10% formalin, paraffin embedded, cut into 5 μm sections and stained with haematoxylin & eosin (H & E) for evaluation.

The salt-leached PLGA was less dense than the hard, dense consistency of the fiber-spun PLGA. This was evidenced by the subsequent cutting of the tissue/PLGA blocks for histological evaluation. The salt-leached PLGA was brittle and prone to crumbling. The fiber-spun PLGA was easy to section as it had a solid consistency and did not crumble.

Histological examination showed a consistent pattern for all specimens in their respective groups. In the salt-leached PLGA group, considerable invasion into the PLGA by microvasculature and new tissue was found throughout, with numerous India Ink filled microvessels evident. The fiber-spun PLGA differed in character. The neovascularization and new tissue formation developed predominantly in a two dimensional plane. Initially, instead of invading the PLGA, tissue preferentially surrounded the PLGA discs and migrated towards the edge of the chamber. Tissue invaded the matrix at a much slower rate. Once the edge of disc was reached further thickening of new tissue grew around the disc but not completely engorging it after 4 weeks.

Further modifications to the fibre-spun PLGA, such as increasing the pore size and decreasing the density (and therefore the hardness) may make this technique a viable alternative to the salt-leached PLGA preparation.

### EXAMPLE 6

### Model System for Vascularized Tissue

The tissue chamber and graft system of the invention may be used as a model to examine the behaviour of vascularised tissue, through the use of an extracorporeal circulation machine to maintain the developing tissue in vitro during its generation. The chamber contents are established as specified in Example 1. The host's blood or suitable transfused blood (at least 90 ml) is taken and heparinised (up to 50 units/ml). The blood vessel ends are connected to silicone tubing and the blood is oxygenated via a renal dialysis filter. The oxygenated blood is pumped through the tissue using conventional intensive care unit instrumentation adapted for this purpose, and maintained in vitro in this manner until the tissue/organ is mature. During this phase blood samples are constantly monitored to assess the degree of coagulation and the maintenance of haemostasis. In a similar manner to the in vivo studies, genetic modification of the tissue generating cells can be applied to this model. Finally the tissue/organ generated is microsurgically replaced into the appropriate site in the host. A major advantage of this method is the ability to produce tailor-made, off-the-shelf parts and organs.

The next step in testing our model is to add stem cells to the system and see whether tissue is generated de novo. The isolation, expansion and seeding of “stem cells” into the chamber is a huge area for research in itself and is still in its infancy. For various reasons, we have chosen an unorthodox method of adding stem cells and environmental cues, with unexpected results. We have investigated the behaviour of injured/necrotic tissue explants placed in vivo in the chamber, and have demonstrated conversion of muscle into fat (see Example 3).

The hypothesis being tested in experiments such as these is that these small tissue explants may harbour at least a few stem cells, which perceive an injury to their parent organs and respond by initiating tissue renewal. We have also tested a number of tissues, including fat, liver and kidney, and will shortly investigate neural, uterus, ovarian, thyroid and glandular tissue. The results have been very promising, because all of the tissues tested have “driven”, by unknown mechanisms, the generation of a cell phenotype not normally present in the chamber. Mechanistically they have converted the cellular/angiogenic response in the chamber from one analogous to “inflammation and scar formation”, involving the de novo generation of tissue largely composed of fibroblasts, to one analogous to “tissue renewal and generation”, also known as “scarless” tissue repair in the fetus, comprising the generation of vascularised tissue with a recognisable three dimensional organisation and phenotype. Significantly, the new tissue formed is free of fibroblastic in-growth and of inflammatory cells.

### EXAMPLE 7

### Assessment of Hypoxia Within the Tissue Growth Chamber

For the study of hypoxia of the cells within the chambers, AV shunt loops were created in anaesthetised male rats as previously described in Example 1. Standard-sized chambers (0.5 ml volume) were used. Chambers were filled with Matrigel, as described in Example 5, and seeded with immortal rat L6 myoblasts (1×10⁶ cells/0.5 ml Matrigel) distributed over the entire surface area. Chambers were then positioned in the groin of the rat.

Chambers were harvested at 3 days, 7 days, and 2 and 4 weeks incubation. At the time of exploration the animals were again an sthetised with sodium phenobariton (30 mg/ml) and an assessment of anoxia was made by injection of nitroimidazole (60 mg/kg, i.p.) 2 hours before the time of chamber harvest: Rats were sacrificed with a lethal dose of pentobarbitone sodium (3 ml of a 325 mg/ml solution) after harvesting the chambers. Specimens within the chambers were processed for histology and immunostaining with nitroimidazole antibody. Under these circumstances, the only cells which label are those which are hypoxic (<10 mm Hg) and which are proliferating.

An assessment of the degree of Oxygenation of tissue at days 3 and 7 showed proliferating, hypoxic cells in the immediate vicinity of the vascular loop at both time points. After 2 weeks the only labelled cells were at the periphery of the growing mass of new tissue. By 4 weeks, no cells were labelled with nitroimidazole.

The results from this study indicate that a state of hypoxia and active biosynthesis exists in cells close to the blood vessel loop. This strongly suggests that hypoxia is a driving force of angiogenesis in the polycarbonate chamber particularly in the first week. Those cells remote from the AV loop were undoubtedly hypoxic but were not proliferating. During week 2 the hypoxic, proliferating cells were located in the advancing edge of the new tissue, but by the end of week 4 the chamber was well oxygenated throughout and new tissue formation had slowed considerably. Studies such as this enable the researcher to invetigate how hypoxia can influence the growth of new tissue within the chamber.

### EXAMPLE 8

### Isolated, Cultured Cells Added to Chambers in the Rat AV Loop Model

(a) Addition of Myoblasts to Chambers

Skeletal muscles from various parts of the body (eg. gastroenemius, rectus femoris, latissimus dorsi, etc) were harvested from neonatal rats 5 days after they were weaned. Myoblasts were generated from this harvested tissue by collagenase digestion and culturing in Ham's F10 culture medium containing 20% fetal calf serum with 2 ng/ml of bFGF. Myoblasts were identified by desmin immunostaining. Fibroblasts were removed by serial subculturing, taking advantage of the fact that they adhere to plastic within half an hour whereas myoblasts adhere after that time. Enriched myoblasts (2-4×10⁶ cells) were inserted into either (1) Matrigel alone (approximately 0.5 ml) or (2) Matrigel (approximately 0.15 ml) with PLGA making up the balance of the volume. These matrices were placed around an AV loop within a standard 0.5 ml chamber, as previously described. These constructs were incubated subcutaneously for either 2, 4, 6, 12 or 16 weeks. At the time of exploration, the rats were placed under general anaesthesia, and the tissue formed within the chamber (also known as the “flap”) was removed. Approximately half of the tissue was frozen in isopentane and the other half fixed in formalin, and sectioned, prior to morphological, histological and immunohistochemical staining.

1. Matrigel Only Group

Group A—2 Weeks (n=6)

The chambers from six rats were examined at 2 weeks. There was a large amount of muscle in four of these; and of these, 3 contained identifiable desmin-positive myoblasts and evidence of myotube formation. The other two contained no desmin-positive tissue.

Group B—6 Weeks (n=9)

Of the 9 rats in this group, 2 constructs contained muscle and myotubes, 4 flaps contained no identifiable muscle, and 3 rats died prematurely.

Group C—12 Weeks (n=11)

Of the 11 rats in this group, no constructs contained muscle, 5 flaps contained no muscle but did contain some (as yet identified) tissue, 2 chambers contained no flap (possibly because it slipped out of the chamber) and 3 rats died prematurely.

2. PLGA/Matrigel Group

Group A—2 Weeks (n=3)

No results for this group.

Group B—6 Weeks (n=6)

Of the 6 rats in this group, 2 constructs contained muscle and myotubes, and 4 flaps contained no muscle. In one chamber in which the myoblasts were fluorescently labelled with CFDA prior to being seeded into the chamber, there was evidence of myoblasts still surviving after 4 weeks' incubation in vivo.

Group C—12 Weeks (n=7)

Of the 7 rats in this group, 2 constructs contained d s-in-stained myoblasts, 5 flaps contained unidentified tissue but no muscle, and 1 rat died prematurely.

Group D—16 Weeks (n=5)

No results for this group.

In H&E stained sections of flaps after 2 weeks incubation, myoblasts were evident in some tissue specimens, with their presence confirmed by immunostaining for desmin. Within 2 weeks, groups of myoblast nuclei had aligned and formed into myotubes which stained positively for dystrophin and formed mature striated skeletal muscle. By 6 weeks, myotubes and mature muscle were present in some specimens but connective tissue formed in others. At both 2, 4 and 6 weeks mononuclear leukocyte infiltrate was present, probably due to the use of Matrigel, which originates from mouse cells. However, by 12 weeks, much of the flap tissue was resorbed. Interestingly, in some of the early experiments with “less pure” myoblasts seeded, isolated pockets of osteoid (bone tissue) and adipose tissue (fat) were also observed after 2 and 4 weeks in the Matrigel only experiments.

In preliminary experiments, a femoral nerve severed distally was incorporated into Matrigel matrix, adjacent to the loop and surrounded by the seeded myoblasts (n=6, 2 weeks incubation). There appeared to be a trend towards reduced desmin-positive muscle cells (compared with the nerve-free controls, Group 1A) but there was positive immunostaining for S100, a Schwann cell marker, in most of the newly generated tissue.

We know from previous work that this model provides a good angiogenic stimulus, and we have mow shown that this model can sustain the survival, expansion and differentiation of myoblasts. The vascularised chamber can also support this cell line and provide an optimal environment in which the chosen cell can differentiate in a normal and expected fashion. Histological evidence demonstrates that the seeded myoblasts both survive and differentiate to form myotubes, which in turn coalesce to form mature skeletal muscle in this model, over a period as short as 2 weeks.

(b) Stem Cell Addition

Using the same AV loop model, we have investigat d the fate of green fluorescent protein (GFP) labelled and non-labelled rat bone marrow-derived stem cells into these chambers.

Bone marrow-derived stromal cells were harvested from rat femurs by flushing them with normal saline. These cells were then labelled and sorted on a FACS machine. The stromal cell subpopulation was expanded by culturing in a α-MEM medium containing 20% fetal calf serum. The expanded cells were retrovirally transfected with Green Fluorescent Protein (GFP) and a neomycin plasmid to enable them to be tracked within our flap. When sufficient cells were available we placed them at a concentration of 2×10⁶ per 0.5 ml Matrigel into our AV loop chamber model.

Nine AV loops in chambers containing these stem cells were constructed using either Matrigel alone (n=8) or Matrigel/PLGA (n=1) and the matrix. Rats have been examined at 2 weeks (n=4) or 4 weeks (n=4). In frozen sections some fluorescence is seen in these specimens, although it is not clear whether this is genuine GFP fluorescence or autofluoresence. In subsequent experiments the resultant tissue from our GFP-labelled flaps has been cultured in the presence of neomycin-rich media. Surviving GFP-labelled cells have been detected under such conditions after 2 and 4 weeks in the chamber, whereas non-GFP-labelled cells failed to survive under these conditions. However, to date we have found no evidence of specific tissue phenotype or clone formation in new tissue arising from these seeded cells.

### EXAMPLE 9

### Pancreatic Cells Added to Chambers in the Rat AV Loop Model to Form a Transplantable Pancreatic Organoid

All experiments were performed using inbred Sprague-Dawley rats. The experimental model used of an arteriovenous (AV) fistula created with a vein graft in the right groin and placed within a 0.5 ml internal volume polycarbonate chamber, was consistent throughout all experimental groups.

Rats were anaesthetised with pentobarbitone prior to surgery as described in previous examples. Pancreatic tissue for transplantation was prepared by various methods:

(a) “Ficoll islets”: Using adult donor rats, the isolated pancreas was digested with collagenase P (Boehringer Mannheim, Germany) in vitro, and the islets purified by centrifugation on a Ficoll density gradient.

(b) “Histopaque islets”: Using adult donor rats, the vasculature of the pancreas was perfused in vivo with 7 ml of collagenase (Worthington Biochemicals, USA) at 1.3 U/ml. The resultant islets were isolated and purified using Histopaque [Liu and Shapiro, 1995].

(c) “Digested pancreas”: Using adult donor rats, the isolated pancreas was digested with collagenase P (Boehringer Mannheim, Germany) in vitro, but the preparation was not subjected to any further purification step.

(d) “Filtered pancreas”: Using adult donor rats, the isolated pancreases were not enzymically digested but simply homogenised and the crude extract sieved through a range of different sized filters. The fraction which passed through the 450 μm filter but was retained by the 100 μm filter was used in further experiments.

The extracellular matrix used as a support for seeding the islet preparations were used in one of the following configurations:

(i) The chamber was filled with Matrigel, and the islets were dispersed throughout.

(ii) The chamber was filled with Matrigel and the islets/pancreatic tissue was placed in centre of chamber/AV loop.

(iii) 150 μl of Matrigel containing the islets/pancreatic tissue was placed in centre of chamber in close proximity to the AV loop.

(iv) 150 μl of rat plasma clot containing the islets/pancreatic tissue was placed in centre of chamber in close proximity to the AV loop.

The experimental groups were devised as follows:

Group 1. Old (400-500 g) inbred Sprague Dawley rats were used. “Ficoll islets” were placed in Matrigel. There were 3 recipient rats. We used a 2.5:1 (donor:recipient) ratio, and 10-17 days incubation.

Group 2. Old (400-500 g) inbred rats were used. “Digested pancreas” were placed in Matrigel. There were 3 recipient rats. We used a 1:1 (donor:recipient) ratio, and 11 days incubation.

Group 3. Adult (230-260 g) inbred rats were used. “Digested pancreas” was placed in Matrigel. There were 6 recipient rats. We used a 1:1 (donor:recipient) ratio, and 7-14 days incubation.

Group 4. Adult (230-260 g) inbred rats were used. “Histopaque islets” were placed in Matrigel. There were a recipient rats. We used both 1:1 and 4:1 (donor:recipient) ratios, and 6-21 days incubation.

Group 5. Adult (230-260 g) inbred rats were used. “Filtered pancreas” was placed in a plasma clot. There were 8 recipient rats. We used a 1:2 (donor:recipient) ratio, and 8-24 days incubation.

In Vitro Experiments

Islets were kept in culture in Matrigel, with DM media changes twice weekly, in parallel with the above in vivo experiments to test the longevity of islets in culture. Insulin immunostaining was performed on several such cultures at one and two months with positive staining results.

Serum Insulin Level Measurements

At the time of chamber harvest, blood samples (100 μl) were taken from the loop artery and vein and systemic venous circulation, for measurement of insulin levels by radioimmunoassay for the rat isoform.

Chamber Harvest and Flap Manipulation

Chambers were harvested at the above time points, and tissues were preserved in Buffered Formal Saline and routine histological preparation, followed by paraffin embedding. Histological s ctions were subject d to routine (H&E) and immunostaining (for insulin and glucagon).

In Vitro Culture

Survival of islets was demonstrated to 4 and 8 weeks in culture. H&E and insulin staining showed functional survival at these time points. The islet clusters had begun to dissociate into individual cells and clumps of cells between 4 and 8 weeks.

Serum Insulin Levels

Serum insulin levels in were tested in experimental groups 3 and 4 described above. Venous (outflow) blood exhibited serum insulin levels that were 30-50% lower than those in the arterial (inflow) serum in most animals. In two animals, levels were 40% and 100% higher in the venous system.

Analysis of the Chambers

Tissue in the chambers was divided into four parts and serial sections made. Large amounts of angiogenesis and collagen deposition were confirmed, in keeping with the original model. H&E staining demonstrated occasional islet persistence in all groups, but not in all flaps. Inflammatory infiltrates were present in most flaps, consisting mainly of lymphocytes. Ductal elements were observed in the Group 5 “filtered pancreas” chambers, although no confirmatory immunohistochemistry was performed. Insulin and glucagon immunohistochemistry demonstrated occasional positive staining, particularly for glucagon.

These experiments demonstrate that the AV loop chamber model creates a suitable environment to support the survival of islets in a significant number of the constructs for periods up to 24 days. Insulin and glucagon production was identified by immunostaining in histological sections of tissue during this same period. However, the long term viability of this new “organoid” and its continued insulin production remains to be evaluated.

### EXAMPLE 10

### Increasing the Amount of Tissue in the Rat Model Through the use of Larger Chambers.

(a) Rat Experiment

The amount of tissu produced in the rat using the standard chamber model (˜0.3 ml) is quite substantial in comparison with the animal's body size, and corresponds to a small “breast” or small “organ” within the body. In order to be able to reproduce this finding in the humans it is essential to test the limits of tissue production. This can be don firstly in the rat, through the use of larger volume chambers. Therefore, the aim of this study was to assess whether larger amounts of tissue could be grown over a longer period of time (4-8 weeks) inside larger chambers. In this fashion it is proposed that this method can be used to produce clinically useful amounts of new tissue which, if necessary, could be transferred on its own vascular pedicle to another part of the same individual.

The basic model of the arteriovenous (AV) shunt loop in an enclosed growth chamber has been described in detail in Example 1. The AV shunt was placed within a dome-shaped chamber (FIG. 2). The chamber was made of polycarbonate, had a proximal opening for the pedicle and consisted of abase plate and a lid. It had a base diameter of 17 mm, a centre-of-base to top-of-dome distance of 1.3 mm and an internal volume of 1.9 ml. In contrast, the standard chamber described in previous studies (for instance ales 1 and 2) had a volume of 0.5 ml. The AV shunt was sandwiched between two custom-made disks of PLGA which was used as a matrix to fill the chamber. The PLGA was prepared according to the salt leaching method described by Patrick et al.(1999). Pore sizes between 300-420 nm and a porosity of 80-90% was achieved. The disks were sterilised by four cycles of mechanical stirring for 30 minutes in 100% ethanol, then three times sterile, phosphate buffered saline, before use.

After positioning the lop in the chamber the lid of the chamber was closed and the chamber embedded beneath the inguinal skin and secured with three 6-0 prolene holding sutures. The wound was closed with 4-0 silk sutures. Chambers were harvested from rats under general anaesthesia at 2, 4, 6, and 8 weeks incubation for further analysis (n=6 per group). Th animal was finally killed by an overdose of Lethobarb (3 ml) administered by intracardiac injection.

Whole mount specimens were fixed in buffered formal saline (BFS) and cut into 1 mm slices. Half of these slices, in alternating order, were embedded in paraffin and stained with H&E for histological comparison of the maturity of the newly formed tissue and its vasculature. The other half of these slices were stored in 100% ethanol and used for point counting on a grid to assess the percentage of the newly formed tissue, the remaining PLGA, and the AV loop in the specimen. Every fifth field of 100 points was counted on the front and back of each tissue slice. For this purpose, the slices were dipped in haematoxylin briefly before counting. This enabled newly formed tissue to be readily distinguished from PLGA. The results of point counting on the grid enabled calculation of the percentages of newly formed tissue, remaining PLGA, and AV loop and comparisons of those values at 2, 4, 6, and 8 weeks. Statistical differences between newly formed tissue weight and PLGA weight in time were calculated using Student's t-test with p<0.05 being statistical significant.

Weight and volume measurements: All specimens harvested from the chambers were assessed for volume and weight. The volumes of the specimens, as measured by fluid displacement, was not statistically significant different from the measured weights. The total average weight (equivalent to volume) of the specimens decreased progressively in time. The total average weight % standard deviation (SD) of each group of specimens was 1.07±0.06, 1.03±0.06, 0.96±0.06, and 0.81±0.18 grams, at 2, 4, 6 and 8 weeks, respectively. This resulted in a statistically significant decrease of specimen weight between time points apart 4 weeks or longer, which may be accounted for by the progressive gradual resorption of PLGA matrix.

The amount of PLGA and tissue in the specimen was studied to assess their involvement in the overall decrease in weight of the specimens. All specimen were point counted microscopically with the aid of a grid to determine the percentage of specimen taken up by PLGA or tissue. The decrease in specimen wet weight was attributed to resorption of PLGA. The total average weight of PLGA±SD at 2, 4, 6, and 8 weeks, respectively, was 0.89±0.07, 0.56±0.14, 0.34±0.07, and 0.20±0.09 g. On the other hand the newly formed tissue component of the specimen showed a progressive increase of weight in time. The total average weight of tissue±SD at 2, 4, 6, and 8 weeks, respectively, was 0.13±0.04, 0.42±0.09, 0.57±0.06, and 0.58±0.10 g. The increase in tissue weight was statistically significant over all consecutive time periods, except for the period between 6 and 8 weeks (P<0.05). Over this 6-8 week period, tissue growth reached a plateau, although it also did not decrease as noticed in previous experiments in smaller sized chambers filled with PLGA (Example 5.).

Macroscopic findings: After India-ink injection, neovascularisation could be readily identified during processing of the tissue. New vasculature did not reach the outer edge of the PLGA scaffold at any time point. However, in one serendipitous finding, a chamber was inadvertently left incubating in a rat for 10 months. When harvested, the chamber was totally full of soft connective tissue, which was well vascularised and had patent blood vessels supplying nutrition to the tissue “flap”.

(b) Rabbit Pilot Experiment

Preliminary results from the rat experiments indicated that the larger chambers were able to grow more tissue and for a longer period than the standard chambers. Where the walls of the large chambers were perforated with numerous holes, a further improvement in the rate of new tissue growth, the amount of tissue produced and growth to the edges of the chamber were found [Tanaka Y, 2000, unpublished findings]. These latter conditions approach the optimal conditions for tissue growth in this model. The major aim of this pilot study was to assess whether tissue production could be scaled up in an animal which is 8-10 times the size of a rat, and whether the tissue would maintain its size and shape.

The experimental model used was the basic AV shunt loop in an enclosed growth chamber, however the experimental animal was the New Zealand White rabbit.

Pre-operative analgesia was given in the form of carprofen (1.5 mg/kg, s.c.). New Zealand White rabbits (2.0 to 2.8 kg) were anaesthetised with i.v. pentobarbitone (30 mg/kg) and maintained in a face mask with halothane and oxygen (2.0 L/min). Under sterile conditions a graft of 4-6 cm (rabbits) respectively was harvested from the left femoral vein, and used to create an AV shunt between the proximal ends of the divided right femoral artery and vein. The AV shunt was placed within a dome-shaped chamber, in this case made of polyurethane, with the approximate dimensions 3.0 cm diameter, 2.0 cm high, with an opening for the vessel entry and egress (FIG. 2). In some instances the anatomy of the rabbit permitted the use of an AV pedicle rather than an AV loop, because the small connecting vessels in the surrounding tissue of the pedicle made it a naturally occurring flow-through loop. In this latter example the effect of the AV blood flow was comparable but the operating time and postoperative pain was less. In the usual configuration this chamber had a plurality of small perforations in the chamber walls. Subcutaneous fat in the groin region was used as a source of adipocytes and adipogenic precursor cells.(Zuk et al, 2001). The fat tissue was formed into a crude slurry by injection through an 18 gauge needle. These cells were donated by and implanted into the same rabbit.

The AV shunt loop or pedicle was placed within the chamber, which was filled with a 3-dimensional matrix made of a combination of PLGA which was machined to fit the chamber, Matrigel, Type 1 porcine skin collagen or a similar suitable composition, and the preadipocyte-rich fat tissue slurry. The Matrigel was then allowed to gel. The lid was closed and the chamber embedded beneath the inguinal skin. The wound closed with 4-0 nylon sutures.

Approximately 6-8 weeks later, with the animal again under general anaesthesia, the chamber with its associated blood vessels was removed from the groin and the chamber. Two flaps have been analysed to date.

The tissue in the chamber was removed and its wet weight recorded. The tissue was also be suspended by a fine cotton suture thread and wholly immersed in a beaker of water on a balance. The mass, assuming a density of 1.00 g/ml, is the tissue volume. Specimens were fixed in buffered formol saline (BFS), embedded in paraffin and stained with either ME or Masson's Trichrome (a connective tissu stain).

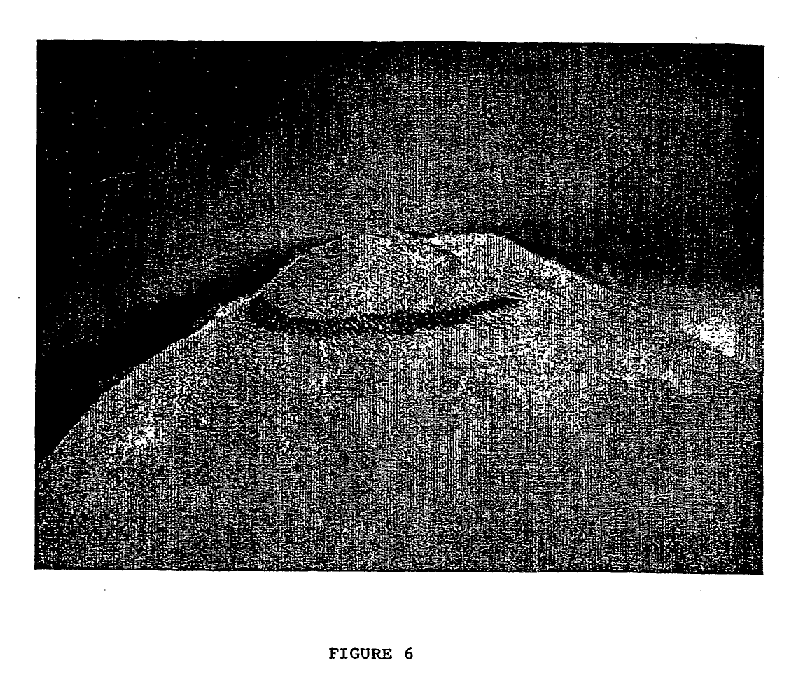

The volume of new tissue generated after 8 weeks growth was 10-11 ml (compared with a total volume of the chamber estimated to be 12 ml). The composition of the flap was adjudged to be a mixture of adipose and other connective tissue. The shape was preserved when transferred under the nipple of the same male rabbit and the volume sufficient to enable the construction of a medium-sized breast on this animal (see FIG. 6).

We have achieved the production of clinically useful amounts of tissue in the rat and rabbit. The tissue thus produced was of a size and shape potentially suitable for breast reconstruction and similar applications. Flaps such as these with their associated patent blood vessels have the potential to be transferred to another part of the body for reconstructive purposes.

### EXAMPLE 11

### A New Model of Vascularised Tissue Engineering in the Mouse

In order to investigate the fundamental processes of tissue engineering it is desirable to develop a suitable tissue engineering model in the mouse for the following reasons:

Genetic Technology: Transgenic and gene knock-out technology is much further advanced in mice, allowing us to probe the influence of a number of factors involved in tissue engineering such as growth promoters and inhibitors.

Stem Cell Biology: Stem cells are pluripotent cells that give rise-to all tissues; they are highly durable and can therefore theoretically resist the initially hostile ischaemic environment of the chamber. This makes them attractive cells to seed in the chamber. Stem cell biologists have cloned a wide variety of stem-cell sub-types in mice that can be seeded into the mouse model in order to attempt to generate specific tissue types.

Cost: There are also significant cost benefits in using mice. Purchase, housing and caring for mice is less expensive than for larger animals. Also there will be a reduction in the use of expensive laboratory consumables such as growth factors.

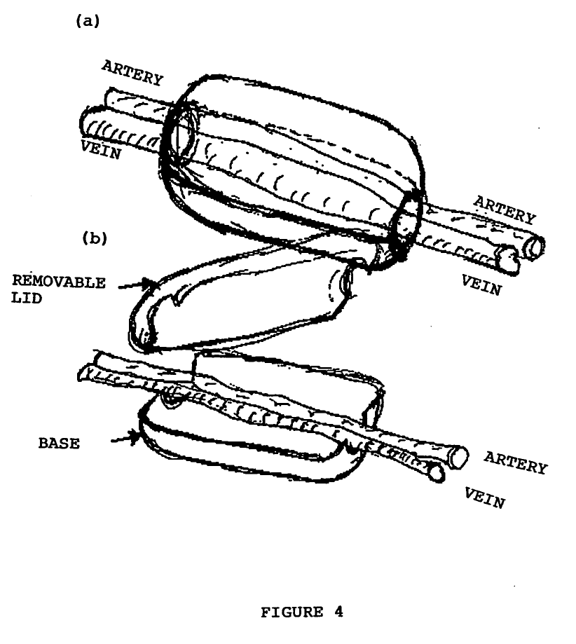

We investigated two different types of vascular configurations that have been shown to be angiogenic in previous work, in order to determine the best technique to use in the mouse. The first was a tied off arteriovenous pedicle (AVP) of the femoral artery and V in (Khouri et al, 1993; FIG. 3) and the second was a “flow through loop” pedicle (FTLP) configuration (Morrison et al, 1990; FIG. 4).

The polycarbonate chamber, when used in the rat model, did not adversely affect the patency rate of the high-flow microsurgical arteriovenous loop. It was also tolerated well by these animals. However this material is hard and has sharp edges which was felt might affect the patency rate in the mouse due to the lower flow rate of the proposed vascular configurations and smaller diameter vessels in this animal. Therefore polycarbonate chambers were compared with softer silicone chambers in order to determine the most suitable material to use in the construct of the chamber. We also compared the two main extracellular matrices used in the rat model (Matrigel and PLGA) in the mouse to judge which was best with regard to angiogenesis and tissue growth. A total of 88 C57BL/6 wild-type mice (male and female; 18-24 g body weight) were used for this set of experiments.

Initially two vascular configurations were examined using specially constructed polycarbonate chambers. The first was a tied off AVP of the mouse femoral artery and vein as described by Khouri et al [1993] in the rat (FIG. 3). The second was a FTL pedicle comprising the superficial epigastric vessels encapsulated within a modified version of the polycarbonate chamber as described by Morrison et al [1990] (FIG. 4). There were 3 groups of 6 animals for both vascular configurations. Each configuration was examined at the 2, 4 and 6 weeks. The experiment was to be performed using both Matrigel® and PLGA as extracellular matrices (n=2×2×3×6=72).

All operations were performed under general anaesthesia (chloral hydrate, 4 mg/g body weight, i.p.). The right groin and upper leg were rendered hair free using a combination of clipping and a depilatory cream. The skin was decontaminated using an alcohol preparation. The tied off pedicle technique required a vertical incision extending form the groin crease to the knee just offset from the saphenous vessels which are visible through the skin. The saphenous vessels were tied off at the kn e and then dissected free from their accompanying nerve back to the origin of the femoral artery at the inguinal ligament. The flow-through model was performed using a transverse groin incision sited just above the groin fat pad. The superficial epigastric (SE) vessels were dissected free of the surrounding tissue from their origin at the femoral vessels for a distance of approximately 1 cm to their entry into the groin fat pad. Here the vessels course through the fat pad sending nutritional branches to the fat and glandular tissue around them. They then anastomose directly with an ilio-inguinal vessel (a direct branch of the infra-renal aorta) that pierces the abdominal wall at the lateral aspect of the inguinal ligament to enter the fat pad from the lateral side. The entire fat pad is mobilised free of the skin and underlying muscle thus creating a space into which the chamber will alter be introduced. Thus the SE vessels have an arterial input and venous drainage from both sides which we felt would augment the long term patency rate in this model. To our knowledge this is the first time that this vascular arrangement has been described in the mouse. The first cm of the SE vessels (where they are free of the fat pad) is then encapsulated in a modified polycarbonate chamber that is split down one side and the appropriate extracellular matrix (Matrigel or PLGA) is inserted into the chamber. The chamber is then sealed at the proximal end and along the lateral split using melted bone wax (Ethicon bone wax™) taking care not to apply the heated wax directly to the vessels. The seal is augmented by two 10/0 nylon microsutures placed at either end of the lateral split and the whole chamber is anchored to the underlying muscle near the origin of the SE vessels in order to prevent the pedicle from being dislodged during post-operative mobilisation. A small amount of fatty tissue surrounding the vessels as they enter the fat pad is allowed to “plug” the distal end of the chamber. This plug is them augmented with wax sealant and the whole construct is carefully plac d in the groin so that it lies in the dissected space lateral to the femoral vessels. The wounds were closed using a combination of buried interrupted horizontal mattress sutures and a running suture (both 6/0 silk) as these animals tend to gnaw at their wounds.

Following early analysis of the results in each group the tied off AVP group of the experiment was discontinued. This was because the thrombosis rate was unacceptably high (11/14 animals) and pursuit of this line of investigation seemed futile and wasteful of animals. This observation contrasts with Khouri's work in the rat and our own experience in the rat and the rabbit where the tied-off AVP remains patent in the majority of cases. The most likely reason for the high thrombosis rate in our study is that the mouse vessels are extremely small (internal diameter approximately 0.2 mm) and very sensitive to dissection. Flow rates in vessels of this size are also very low. The thrombosis rate in the FTLP group was better (3/11) but still seemed excessively high.

We postulated that the polycarbonate material we were using was too hard and sharp for the delicate vessels of the mouse. Our experimental plan was modified to include a cohort of animals with chambers made from medical grade silicone (Animal Ethics committee approval was obtained for this modification). Two cohorts (1 with PLGA and 1 with Matrigel®) of 3 groups of 4 animals were used for this modified aspect of the experiment (n=2×3×4=24) using only the flow through vascular configuration. Accurate estimation of the volume and weight of the specimens proved impossible. The volume of the chamber is approximately 80-100 μl. This varies for several reasons such as the amount of wax or fat that encroaches on the entry points of the chamber. Also it is difficult to measure the exact volume of extracellular matrix that is used in each chamber. Matrigel is usually added as a liquid and allowed to gel in vivo. Some spillage may occur during infusion or during manipulation of the chamber. We also noted that the volume of the Matrigel declined by at least 50% over the first two weeks such that the specimen that was removed was actually smaller than that inserted.

The weight of the PLGA used in each chamber could be accurately measured but the volume was impossible to ascertain as the structure was porous and had to be broken up into crumbs in order to easily fit it into the small chamber. Given these inaccuracies we did not attempt to evaluate quantitatively the chamber tissue and looked at the more qualitative aspects of the device such as morphology of the newly formed tissue. Patency of the vessels was determined at microsurgical exploration and via India ink perfusion studies. If the vessel was extensively thrombosed within the chamber it was usually possible to see this under the operating microscope. However ascertaining definitive patency was not always possible. Therefore India ink perfusion studies were performed under general anaesthesia on each animal prior to sacrifice. Under the operating microscope the groin incision was reopened and the chamber exposed taking care not to damage the pedicle. A laparotomy was then performed and the abdominal aorta was dissected free of the vena cava and cannulated just below the renal vessels using a fine (30G) bore silicone tube. This was then flushed using heparinised saline to ensure that the cannula was in the correct position. Next a solution of neat commercial India ink containing 10 i.u. heparin per ml was infused under gentle hand pressure using in a pulsatile fashion until the animals liver had turned completely black. Previous descriptions also advise the use of gelatin in this solution but in our preliminary trials of the technique we found that the gelatin formed clumps that obstructed the fine bore tube and resulted failure of the procedure (this occurred even if the gelatin was warmed to body temperature prior to infusion). Patency could be confirmed under direct visualization of the transparent chamber as the India ink could clearly be seen tracking into the chamber along the vascular pedicle. Following this the animals were sacrificed via a lethal overdose of phenobarbitone and the chambers were carefully removed cutting the pedicle(s) flush with their entry into the device.

The specimens were fixed in formalin and taken through graded alcohol solutions to absolute alcohol. They were then immersed in methyl salicylate and allowed to clear over 72 hours. This allows direct visualization of the vascular tree which has been perfused with India ink. All specimens were then examined as whole-mount preparations under microcater and vessel counts were performed. After this the specimens were processed for histological examination and embedded in wax. The wax blocks were then sectioned at 5 μm and stained with haematoxylin and eosin in a standard fashion. Vessel area density was estimated on all cleared specimens using a microcater which allowed visualization throughout the depth of these small tissue specimens. Three fields were randomly selected at 3 depth intervals of 500 μm and the vessel density was assessed with the aid of a stereometric grid. Following completion of this process the specimens were committed to histological processing. The stained sections were morphologically assessed in terms of angiogenesis and the cellular characteristics of the newly generated tissue. Univariate analysis of the patency rates and vessel density was performed using the Student t-test. The patency rate was assessed for the two vascular configurations and for the different materials used in the make-up of the chamber.

The patency rate for the tied off arteriovenous pedicle was 21% versus 88% for the flow-through pedicle. The patency rate in the polycarbonate chambers (excluding the tied off AV pedicle group) was 88% versus 97% in the silicone chambers. The new vessels in the tied off AVP group were seen to be arising from outside the chamber and growing in along the thrombosed pedicle. The vessel densities in the flow-through chambers were similar at 2, 4 and 6 weeks. Similarly there was no difference in vessel density between PLGA and Matrigel. Morphologically there was good angiogenesis in Matrigel® and PLGA but qualitatively it was better in the Matrigel®. The new vessels seemed to be more numerous and occurred throughout the construct in the Matrigel®. The angiogenesis in the PLGA was more to the periphery of the construct with fewer vessels in the central aspect probably due to the solid nature of this ECM.

In terms of cellular morphology the PLGA seemed to promote a predominately fibrous foreign-body type reaction. Fibroblasts are the predominant cell s en both peripherally where the matrix lay against the chamber wall and centrally within the substance of the matrix. The Matrigel® group also showed a fibroblastic response at the ECM-chamber interface. On the other hand the central aspect of the Matrigel® shows the presence of fat in the chamber that has clearly migrated through the matrix and survived, presumably nourished by the newly generated vascular tree. This phenomenon has been reported before in non-encapsulated Matrigel® in mice using growth factors and pre-adipocytes. The presence of mature viable fat in the chamber suggests this model is capable of supporting the migration, maturation and possibly the reproduction of fat cells and their precursors. In female animals the fat pad contains some mammary tissue and associated ducts which are occasionally found in the distal part of the chamber where this tissue is used as a “plug” to seal the distal aperture. In the Matrigel group we observed that in some of these animals the breast ductal/acinar tissue seemed to be growing into the Matrigel and in others there is clear morphological evidence of newly forming ductal/acinar tissue. This suggests that the chamber is capable of supporting the development of glandular tissue as well as fat. To our knowledge this has not been reported before.

We have seeded the chamber with clones of mouse mesenchymal stem cells that were cultured from a C57 Immorto mouse and also with a mouse mammary tumour cell line. Both were labelled with flourescent markers (GFP or CFDA) and we were able to demonstrate that the implanted cells were alive at 48 hours, 4 days, 2 weeks, 4 weeks and 8 weeks. The mammary tumour line has been seen histologically at 4 weeks demonstrating that the chamber is capable of supporting cell lines in the longer term. We have also successfully grown foetal pancreas, liver, heart, bowel and limb bud (composite skin, bone, cartilage, muscle, vessel and nerve) in immunodeficient SCID mice. As well as this we have been successful in getting cultured adult pancreatic islets to survive and produce hormones at 2 and 10 weeks in wild type mice (C57BL6). This effectively means that we have successfully grown functioning islet allograft in these animals which has not been achieved in other models of pancreatic transplantation. This means that the chamber may confer some immuno-privileged status to the cells that grow within it. This has therapeutic implications in that it may be possible to use unmatched allograft or even xenograft in the chamber with or without local immunosuppression or Sertoli cell co-culture as a treatment of Diabetes Mellitus.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

References cited herein are listed on the following pages, and are incorporated herein by this reference.

### REFERENCES

Erol, Ö. O., and Spira, M. Surgery (1980) 66: 109-115.

Khouri, R. K., Koudsi, B., Deune, E. G., Hong, S. P., Ozbek, M. R., Serdar, C. N., Song, S-Z and Pierce, G. F. Surgery (1993) 114:374-380.

Knight, K. R., Mian, R., Tanaka, Y., Penington, A. J., Hurley, J. V., Cassell, O., Romeo, R., and Morrison, W. A. 7th Annual Meeting Australian Vascular Biology Society (1999).

Liu, M., and Shapiro, M. E. Transplant. Proc. (1995) 27: 3205-3207.

Morrison, W. A., Dvir, E., Doi, K., Burley, J. V., Hickey, M. J., and O'Brien, B. M. Br. J. Plast. Surg. (1990) 43: 645-654.

Patrick, C. W. Jr., Chauvin, P. B., Hobley, J., and Reece, G. P. Tissue Eng. (1999) 5: 139-151.

Prockop, D. J. Science (1997) 276: 71-74.

Tanaka, Y., Tajima, S., Tsutsami, A., Akamatsu, J., and Ohba, S. J. Jpn. P.R.S. (1996) 16: 679-686.

Tanaka, Y., Tsutsumi, A., Crowe, D. M., Tajima, S., and Morrison, W. A. Br. J. Plast. Surg. (2000) 53: 51-57.

Vukicevic, S., Kleinman, H. K., Luyten, F. P., Roberts, A. B., Roche, N. S. and Reddi, A. B. Exp. Cell Res. (1992) 202: 1-8.

Zuk, P. A., Zhu, M., Mizuno, K., Huang, J., Futrell, W., Katz, A. J., Benhaim, P., Lorenz, H. P., and Hedrick, M. H. Tissue Eng. (2001) 7: 211-228.

The claims defining the invention are as follows:

1. A method of producing donor vascularised tissue, suitable for transplantation into a recipient animal in need of such treatment, comprising the steps of:
a) creating a functional circulation on a vascular pedicle in a donor subject;
b) partially or totally enclosing the vascular pedicle within a fabricated chamber;
e) seeding the chamber with isolated cells or pieces of tissue;
d) implanting the chamber containing the vascular pedicle into donor subject at a site where such an anatomical construct can be created; and
e) leaving the chamber in the implantation site for a period sufficient to allow the growth of vascularised new tissue.

2. A method according to claim 1, comprising the step of: after step (a) surrounding the vascular pedicle with added extracellular matrix and/or a mechanical support

3. A method according to claim 1 or claim 2, comprising the step of: after step (b) adding growth factors, drugs, antibodies, inhibitors or other chemicals to the chamber.

4. A method according to claim 1, in which the vascular pedicle comprises an arterio-venous (AV) loop or shunt.

5. A method according to claim 1, in which the vascular pedicle compris s a ligated artery and vein.

6. A method according to claim 1, wherein the chamber in step (e) is left in the implantation site for at least 4 weeks.

7. A method according to claim 1, wherein the chamber in step (e) is left in the implantation site for at least 6 weeks.

8. A method according to claim 1, herein said created vascular pedicle contained within the chamber is connected to an extracorporeal circulation.

9. A method according to claim 1, wherein the donor subject of step (a) is a mammal.

10. A method according to claim 9, wherein said mammal is a human.

11. A method according to claim 1, comprising the additional step of implanting said vascularised new tissue into an autologous recipient.

12. A method according to claim 1, comprising the additional step of implanting said vascularised new tissue into a heterologous recipient.

13. A method according to claim 2, wherein the added extracellular matrix is selected from the group consisting of reconstituted basement membrane preparations, polylactic-polyglycolic acid variants (PLGA), fibrin or plasma glue, and native collagen.

14. A method according to claim 13, wherein said PLGA comprises PLGA sponge.

15. A method according to claim 3, wherein the additional growth factors, drugs, antibodies, inhibitors or other chemicals added to the chamber are selected from the group consisting of growth factors, sing factors to attract stem cells from the circulation, exogenous factors and promoters of angiogenesis or vasculogenesis.

16. A method according to claim 1, wherein the isolated cells or pieces of tissue of step (c) are autologous to the host.

17. A method according to claim 1, wherein the isolated cells or pieces of tissue of step (c) are heterologous to the host.

18. A method according to claim 1, wherein the isolated cells or pieces of tissue of step (c) are selected from the group consisting of stem cells, skeletal muscle tissue that has been subjected to ischaemia, myoblasts transfected with Myo-D, keratinocytes, myoblasts, fibroblasts, pre-adipocytes, adipocytes, cardiomyocytes, endothelial cells, smooth muscle cells, chondrocytes, pericytes, bone-marrow derived stromal precursor cells, Schwann cells and other cells of the peripheral and central nervous system, olfactory cells, hepatocytes and other cells of the liver, mesangial cells and other cells of the kidney, pancreatic islet β-cells and ductal cells, thyroid cells, cells of other endocrine organs, portions of skeletal or cardiac muscle, pancreas, liver, epididymal and other subcutaneous fat, nerves, kidney, bowel, ovary, uterus, testis, and glandular tissue from endocrine organs.

19. A method according to claim 1, wherein the functional circulation on a vascular pedicle of step (a) comprises an artery and a vein.

20. A method according to claim 1, wherein the functional circulation on a vascular pedicle of step (a) comprises an artery, a venous graft, and a vein.

21. A method according to clam 1, wherein the functional circulation on a vascular pedicle of step (a) comprises an artery, a venous graft, an arterial graft, and a vein.

22. A method according to claim 1, wherein the functional circulation on a vascular pedicle of step (a) comprises the ligated stumps of an artery and a vein placed side by side.

23. A vascularised tissue graft produced by a method according to claim 1.

24. A method of repairing a tissue deficit, comprising the steps of:
a) creating a vascularised tissue graft according to claim 23;
b) retaining the graft in the donor subject for a sufficient period to produce tissue with the desired size, vascularity and degree of differentiation;
c) transferring the graft to the desired recipient site; and
d) anastomosing the blood vessels of the graft to a local artery and vein.

25. A method of tissue augmentation, comprising the steps of:
a) creating a vascularised tissue graft according to claim 23;
b) retaining the graft in the donor subject for a sufficient period to produce tissue with the desired size, vascularity and degree of differentiation;
c) transferring the graft to the desired recipient site; and
d) anastomosing the blood vessels of the graft to a local artery and vein.

26. A method of delivery of a gene product to a subject, comprising the steps of:
a) creating vascularised tissue in a tissue chamber according to the method of claim 1;
b) removing the chamber with its vascularised tissue and culturing the chamber assembly in vitro;
c) transforming cells of the tissue in the clamber with a desired gene; and
d) implanting the vascularised tissue with or without the chamber into a patient in need of such treatment.

27. A model system for vascularised tissue, comprising a tissue chamber comprising an isolated vascular pedicle produced by a method according to claim 1, wherein the tissue chamber is operably connected to an extracorporeal circulation apparatus and to a renal dialysis filter.
